(19) United States
(12) Patent Application Publication
(10) Pub. No.: US 2003/0049203 A1
(43) Pub. Date: Mar. 13, 2003

(54) Targeted nucleic acid constructs and uses related thereto

(76) Inventors: David R. Elmaleh, Newton, MA (US); Alan J. Fischman, Boston, MA (US); John W. Babich, Scituate, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 09/945,166

(22) Filed: Aug. 31, 2001

(51) Int. Cl. A61K 51/00; A61K 38/16; A61K 49/00; A61K 48/00; A61K 39/395; C07K 16/46

(52) U.S. Cl. 424/1.73; 514/8; 424/178.1; 514/44; 424/9.6; 536/23.2; 530/389.1; 530/395

(57) ABSTRACT

The invention provides targeted constructs comprising a targeting moiety, a nucleic acid, and a payload. The payload can be a detectable label or a therapeutic agent. The nucleic acid can be an antisense molecule that is complementary to RNA present in a target cell. The targeted constructs can be used to introduce the payload into a target cell in vivo or in vitro. Accordingly, the invention can be used for diagnostic purposes and for therapeutic purposes.

### GOVERNMENT SUPPORT

Work described herein was supported in part by funding from the Department of Energy under DOE grant DE-FG02-86ER60460.

### BACKGROUND OF THE INVENTION

As the importance of genes as therapeutic and diagnostics has emerged, the use of nucleic acids in diagnostic and therapeutic applications has blossomed. Nucleic acids can be used as probes to detect the presence or absence of gene expression, or to discern mutations associated with disease states. Nucleic acids can also be used as antisense therapeutic agents which inhibit the expression of target genes. Furthermore, vectors can be used to insert or delete genes in cells, thereby changing the genotype of the affected cells. Recently, nucleic acids have been joined to other molecules to enhance the effectiveness of a therapeutic agent.

U.S. Pat. No. 4,904,582 to Tullis, U.S. Pat. No. 5,420,330 to Brush, U.S. Pat. No. 5,834,607 to Manoharan, U.S. Pat. No. 5,223,263 to Hostetler et al., and U.S. Pat. No. 5,763,208 to Bischofberger, as well as international applications WO 96/07392, WO 90/10448, and WO 96/18372 disclose nucleic acids linked to hydrophobic/lipophilic moieties which enhance membrane transport of the construct. A wide variety of charged and uncharged lipids and derivatives thereof are known to facilitate intermembrane transport of exogenous molecules.

U.S. Pat. No. 5,852,182 and 5,578,718 to Cook et al., and U.S. Pat. No. 5,414,077 to Lin et al. present thiol-derivatized oligonucleotides. The thiol moieties are used to link the oligonucleotides to other molecules, such as peptides, proteins, lipophilic molecules, steroids or reporter molecules.

U.S. Pat. No. 5,510,475 to Agrawal, et al., discusses modified oligonucleotides bearing reporter molecules.

International application WO 95/02422 relates to oligonucleotides coupled to antibodies for targeting the oligonucleotide to a specific cell.

U.S. Pat. No. 5,514,786 to Cook et al. describes constructs which include a nucleic acid, an intercalating moiety, and a reactive portion that contributes to or effects the cleavage of RNA.

U.S. Pat. No. 5,830,658 to Gryaznov discloses branched polymers which include an oligonucleotide as a target binding moiety and a signal-generating moiety capable of generating a detectable signal, such as biotin.

U.S. Pat. No. 5,820,847 and 5,688,488 to Low et al., and U.S. Pat. No. 5,716,594 to Elmaleh et al. discuss linking nutrients such as folate, biotin, and riboflavin to molecules such as nucleic acids to facilitate their uptake by cells expressing the corresponding receptors, especially tumor cells and sites of infection.

However, none of the above constructs uses a non-oligonucleotide molecule to target an oligonucleotide and a therapeutic or diagnostic agent, thereby permitting faster, specific delivery of a therapeutic or diagnostic agent to target cells. Simple, rapid methods for more specifically localizing therapeutic or imaging agents within target cells, such as tumors and sites of infection, in vivo are needed, particularly methods which promote the retention of the agents in the target cells.

### SUMMARY OF THE INVENTION

The present invention relates to nucleic acid constructs comprising at least three portions: a targeting moiety (T.M.), a nucleic acid (N.A.), and a payload. These constructs may be used to specifically direct a diagnostic, therapeutic, or other payload to a desired location in an organism or in cells in vitro.

In one aspect, a targeted oligonucleotide construct includes a targeting moiety which localizes to a site in an organism, an oligonucleotide complementary to a nucleic acid of interest, and a detectable label. The site in the organism may be the location of an abnormal physiological condition, or a particular tissue type. The targeting moiety may be a lipid, antibody, lectin, ligand, sugar, steroid, hormone, nutrient, or protein. The detectable label may be a chemiluminescent label, a radioisotope, a fluorescent label, a paramagnetic contrast agent, or a metal chelate. The oligonucleotide may be an antisense oligonucleotide or an antisense oligonucleotide analog. In one embodiment, the detectable label and the targeting moiety are coupled to the oligonucleotide. In another embodiment, the oligonucleotide and the detectable label are coupled to the targeting moiety. In yet another embodiment, the targeting moiety and the oligonucleotide are coupled to the detectable label.

In another aspect, a targeted oligonucleotide conjugate comprises a targeting moiety which localizes to a site in an organism, an oligonucleotide complementary to a nucleic acid of interest, and a therapeutic agent. The targeting moiety may be a lipid, antibody, lectin, ligand, sugar, steroid, hormone, nutrient, or protein. The oligonucleotide may be an antisense oligonucleotide or an antisense oligonucleotide analog. The therapeutic agent may be an enzyme, an enzyme inhibitor, a receptor ligand, a radioisotope, an antibiotic, a steroid, a hormone, a polypeptide, a glycopeptide, a phospholipid, or a drug. In one embodiment, the detectable label and the targeting moiety are coupled to the oligonucleotide. In another embodiment, the oligonucleotide and the detectable label are coupled to the targeting moiety. In yet another embodiment, the targeting moiety and the oligonucleotide are coupled to the detectable label.

In another aspect, the invention provides a method for preparing a targeted oligonucleotide construct, by forming a conjugate by connecting a targeting moiety which localizes to the site of an abnormal physiological condition to an oligonucleotide complementary to a nucleic acid of interest, and connecting a detectable label to the conjugate. In another embodiment, the method includes forming a conjugate by connecting a targeting moiety which localizes to the site of an abnormal physiological condition to a detectable label, and connecting to the conjugate an oligonucleotide complementary to a nucleic acid of interest. In yet another embodiment, the method includes forming a conjugate by connecting a detectable label to an oligonucleotide complementary to a nucleic acid of interest, and connecting to the conjugate a targeting moiety which localizes to the site of an abnormal physiological condition.

In a related aspect, the invention provides a method for preparing a targeted oligonucleotide construct by forming a conjugate by connecting a targeting moiety which localizes to the site of an abnormal physiological condition to an oligonucleotide complementary to a nucleic acid of interest, and connecting a therapeutic agent to the conjugate. In another embodiment, the method includes forming a conjugate by connecting a targeting moiety which localizes to the site of an abnormal physiological condition to a therapeutic agent, and connecting to the conjugate an oligonucleotide complementary to a nucleic acid of interest. In yet another embodiment, the method includes forming a conjugate by connecting a therapeutic agent to an oligonucleotide complementary to a nucleic acid of interest, and connecting to the conjugate a targeting moiety which localizes to the site of an abnormal physiological condition.

The invention further provides a method for treating a physiological condition in a patient by administering an amount of a targeted construct sufficient to treat the physiological condition. Furthermore, the invention provides a method for imaging a physiological condition in a patient, by administering to the patient a targeted construct including a detectable label, and detecting the label in the patient.

### BRIEF DESCRIPTION OF THE FIGURES

FIG. 1 show representations of structures of targeted constructs, which are comprised of nucleic acid molecule (N.A.), a targeting moiety (T.M.), and a payload.

FIG. 2A shows the chemical synthesis scheme 1.

FIG. 2B shows the chemical synthesis scheme 2.

FIG. 3 is a high pressure liquid chromatography (HPLC) diagram of ¹²⁵I-c-myb antisense (dotted line) and sense (continuous line) phosphorothioate oligonucleotides after storage at −20° C. for 6 months.

FIG. 4A shows the amount of ¹²⁵I-c-myb phosphorothioate antisense (top line) and sense (lower line) oligonucleotides by HISM cells as a function of time.

FIG. 4B shows the amount of ¹²⁵I-c-myb phosphorothioate antisense (top line) and sense (lower line) oligonucleotides by SK-N-SH cells as a function of time.

FIG. 4C shows the amount of ¹²⁵I-c-myb phosphorothioate antisense (top line) and sense (lower line) oligonucleotides by NIH-3T3 cells as a function of time.

FIG. 5A shows the % uptake of ¹²⁵I-c-myb phosphorothioate antisense (top line) and sense (lower line) oligonucleotides by HISM cells per 10⁵ cells as a function of the concentration of the oligonucleotide added to the cell culture. The data are normalized to percent of total applied activity per 10⁵ cells.

FIG. 5B shows the % uptake of ¹²⁵I-c-myb phosphorothioate antisense (top line) and sense (lower line) oligonucleotides by SK-N-SH cells per 10⁵ cells as a function of the concentration of the oligonucleotide added to the cell culture. The data are normalized to percent of total applied activity per 10⁵ cells.

FIG. 5C shows the % uptake of ¹²⁵I-c-myb phosphorothioate antisense (top line) and sense (lower line) oligonucleotides by NIH-3T3 cells per 10⁵ cells as a function of the concentration of the oligonucleotide added to the cell culture. The data are normalized to percent of total applied activity per 10⁵ cells.

FIG. 6 shows the uptake of ¹²⁵I-c-myb phosphorothioate antisense (plaid columns) and sense (plain columns) oligonucleotides or a mixture of the two (last column) (as an amount of radioactivity per well) by SK-S-NH cells incubated with the indicated concentrations of the oligonucleotides for 20 seconds (first two columns) or 40 minutes.

FIG. 7 shows the uptake and retention of ¹²⁵I-c-myb phosphorothioate antisense and sense oligonucleotides by HISM, SK-N-SH and NIH-3T3 cells incubated for 60 or 120 minutes with the oligonucleotide, followed by a wash and incubation in medium without oligonucleotide for 30 minutes (i.e., washout).

FIG. 8 shows the amount of ¹²⁵I-c-myb phosphorothioate antisense oligonucleotide present in various organs of rats at various times after injection (“p.i.”) of the rats with the oligonucleotide (“biodistribution”), presented as percent injected dose per gram. Each value is the mean±sem for 6 animals.

### DETAILED DESCRIPTION OF THE INVENTION

The present invention provides nucleic acid constructs comprising at least three portions: a targeting moiety (T.M.), a nucleic acid (N.A.), and a payload. The targeting moiety may be any molecular structure which assists the construct in localizing to a particular target area, entering a target cell(s), and/or binding to a target receptor. The nucleic acid may be an oligonucleotide selected to be complementary to a nucleic acid (e.g., DNA, RNA, etc.) known or suspected to be active or present in the target area or target cells. For example, the nucleic acid may be an antisense oligonucleotide complementary to RNA or DNA of a virus suspected of infecting cells, to a nucleic acid expressed in certain types of tumor cells, or any other nucleic acid associated with an abnormal condition or a tissue type. The payload may be a therapeutic agent (e.g., a drug, a radiotherapeutic atom, etc.), a detectable label (e.g., fluorescent, radioactive, radiopaque, etc.), or any other agent desired to be delivered to a site or cell type in vivo or in vitro, e.g., a site of an abnormal condition or tissue type. Preferred complexes are sufficiently stable to prevent significant uncoupling prior to internalization by the target cell. However, the complex may be clearable under appropriate conditions within the target cell. A targeted construct may include more than one payload, e.g., a therapeutic agent and a detectable label, a drug and a radiotherapeutic atom, etc. The various portions of the targeted constructs will be discussed in greater detail below.

Definitions

As used herein, the following terms and phrases shall have the meanings set forth below.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

“Antisense” nucleic acids refer to nucleic acids that specifically hybridize (e.g., bind) with a nucleic acid, e.g., cellular mRNA and/or genomic DNA, under cellular conditions so as to inhibit expression (e.g., by inhibiting transcription and/or translation). The binding may be by conventional base pair complementarity or, for example, in the case of binding to DNA duplexes, through specific interactions in the major groove of the double helix.

“Complementary” nucleic acids, as the term is used herein, refers to sequences which have sufficient complementarity to be able to hybridize under highly stringent or mildly stringent conditions, thereby forming a stable duplex. “Completely complementary” nucleic acids refers to nucleic acids having nucleotide sequences in which each base in one nucleic acid is complementary to that in that in the other nucleic acid, permitting base pair formation at each position of complementary sequences of the two nucleic acids.

“Conjugated” shall mean ionically or, preferably, covalently attached (e.g., via a crosslinking agent).

The language “effective amount” of a targeted therapeutic agent or imaging agent refers to that amount necessary or sufficient to eliminate, reduce, or maintain (e.g., prevent the spread of) an infection, tumor, or other target. The effective amount can vary depending on such factors as the disease or condition being treated, the particular targeted constructs being administered, the size of the subject, or the severity of the disease or condition. One of ordinary skill in the art can empirically determine the effective amount of a particular compound without necessitating undue experimentation.

A “folate”, as the term is used herein, refers to folic acid, a derivative or analog thereof, or a related compound that binds to a folate receptor. Folic acid, folinic acid, pteropolyglutamic acid, and folate receptor-binding pteridines such as tetrahydropterins, dihydrofolates, tetrahydrofolates, and their deaza and dideaza analogs are preferred complex-forming ligands used in accordance with this invention. The terms “deaza” and “dideaza” analogs refer to the art-recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, and 5,8-dideaza analogs. Other folates useful as complex-forming ligands for this invention are the folate receptor-binding analogs aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). Other suitable ligands capable of binding to folate receptors to initiate receptor-mediated endocytotic transport of the construct include anti-idiotypic antibodies to a folate receptor.

“Human monoclonal antibodies” or “humanized” murine antibodies, as the terms are used herein, refer to murine monoclonal antibodies “humanized” by genetically recombining the nucleotide sequence encoding the murine Fv region (i.e., containing the antigen binding site) or the complementarity-determining regions thereof with the nucleotide sequence encoding at least a human constant domain region and an Fc region, e.g., in a manner similar to that disclosed in European Patent Application Publication No. 0,411,893 A3. Some additional murine residues may also be retained within the human variable region framework domains to ensure proper target site binding characteristics. Humanized antibodies are recognized to decrease the immunoreactivity of the antibody or polypeptide in the host recipient, permitting an increase in the half-life and a reduction in the possibility of adverse immune reactions.

An “imaging agent” shall mean a composition capable of generating a detectable image upon binding with a target and shall include radionuclides (e.g., In-111, Tc-99m, I-123, I-125 F-18, Ga-67, Ga-68, and for Positron Emission Tomography (PET) and Single Photon Emission Tomography (SPECT), unpair spin atoms and free radicals (e.g., Fe, lanthanides, and Gd) and contrast agents (e.g., chelated (DTPA) manganese) for Magnetic Resonance Imaging (MRI).

“Nucleic acid” refers to polynucleotides, such as deoxyribonucleic acid (DNA) and, where appropriate, ribonucleic acid (RNA). The term should also be understood to include, as equivalents, analogs of either RNA or DNA made from nucleotide analogs, and, as applicable, to the embodiment being described, single (sense or antisense) and double-stranded polynucleotides. The term encompasses oligonucleotides, e.g., sequences comprised by less than or equal to about 100 bases, more preferably less than about 50 bases, and most preferably less than about 25 bases.

The term “payload” includes therapeutic agents (e.g., a drug, a radiotherapeutic atom, etc.), detectable labels (e.g., fluorescent, radioactive, radiopaque, etc.), or any other moiety desired to be delivered to a target site, e.g., that of an abnormal condition.

A “pharmaceutically acceptable carrier” is intended to include substances that can be coadministered with a targeted therapeutic agent and allows the compound to perform its intended function. Examples of such carriers include solutions, solvents, dispersion media, delay agents, emulsions and the like. The use of such media for pharmaceutically active substances are well known in the art. Any other conventional carrier suitable for use with the targeted constructs also falls within the scope of the present invention.

“Small molecule” refers to a composition which has a molecular weight of less than about 2000 amu, preferably less than about 1000 amu, and even more preferably less than about 500 amu.

“Subject” shall mean a human or non-human animal (e.g., non-human primate, rat, mouse, cow, pig, horse, sheep, ovine, bovine, monkey, cat, dog, goat etc.).

A “target” shall mean an in vivo or in vitro site to which targeted constructs bind. A preferred target is a tumor (e.g., tumors of the brain, lung (small cell and non-small cell), ovary, prostate, breast and colon as well as other carcinomas and sarcomas). Another preferred target is a site of infection (e.g., by bacteria, viruses (e.g., HIV, herpes, hepatitis) and pathogenic fingi (Candida sp.). Particularly preferred target infectious organisms are those that are drug resistant (e.g., Enterobacteriaceae, Enterococcus, Haemophilus influenza, Mycobacterium tuberculosis, Neisseria gonorrhoeae, Plasmodium falciparum, Pseudomonas aeruginosa, Shigella dysenteriae, Staphylococcus aureus, Streptococcus pneumoniae). A target may refer to a molecular structure to which a targeting moiety binds, such as a hapten, epitope, receptor, dsDNA fragment, carbohydrate, or enzyme. Additionally, a target may be a type of tissue, e.g., neuronal tissue, intestinal tissue, pancreatic tissue, etc. Exemplary specific targets are provided below in Tables 1 and 2.

“Target cells” which can serve as the target for the method of this invention include prokaryotes and eukaryotes, including yeasts, plant cells and animal cells, e.g., human cells. The present method can be used to modify cellular function of living cells in vitro, i.e., in cell culture, or in vivo, where the cells form part of or otherwise exist in plant tissue or animal tissue. Thus the cells can form, for example, the roots, stalks or leaves of growing plants and the present method can be performed on such plant cells in any manner which promotes contact of the targeted construct with the targeted cells. Alternatively, the target cells can form part of the tissue in an animal. Thus the target cells can include, for example, the cells lining the alimentary canal, such as the oral and pharyngeal mucosa, cells forming the villi of the small intestine, cells lining the large intestine, cells lining the respiratory system (nasal passages/lungs) of an animal can be contacted by inhalation of the present complexes, dermal/epidermal cells and cells of the vagina and rectum, cells of internal organs including cells of the placenta and the so-called blood/brain barrier, etc.

A “targeting construct” or “targeted construct” refers to a molecular complex comprising a targeting moiety (T.M.), a nucleic acid (N.A.) and a payload. At least two of these elements are preferably covalently bound to each other. A “covalent targeting complex” refers to a targeting complex wherein the targeting moiety, the nucleic acid and the payload are covalently linked to each other, as further described herein.

A “targeted oligonucleotide construct” refers to a targeted construct, wherein the nucleic acid is an oligonucleotide.

The term “targeting moiety” refers to any molecular structure which assists the construct in localizing to a particular target area, entering a target cell(s), and/or binding to a target receptor. For example, lipids (including cationic, neutral, and steroidal lipids, virosomes, and liposomes), antibodies, lectins, ligands, sugars, steroids, hormones, nutrients, and proteins can serve as targeting moieties.

A “therapeutic agent” shall mean an agent capable of having a biological effect on a host. Preferred therapeutic agents are capable of preventing the establishment or growth (systemic or local) of a tumor or infection. Examples include boron-containing compounds (e.g. carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals. In a preferred embodiment for treating or preventing the establishment or growth of a tumor, the therapeutic agent is a radionuclide, toxin, hormone antagonist, heavy metal complex, oligonucleotide, chemotherapeutic nucleotide, peptide, non-specific (non-antibody) protein, a boron compound or an enediyne. In a preferred embodiment for treating or preventing the establishment or growth of a bacterial infection, the therapeutic agent is an antibiotic, radionuclide or oligonucleotide. In a preferred embodiment for treating or preventing the establishment or growth of a viral infection, the therapeutic agent is an antiviral compound, radionuclide, or oligonucleotide. In a preferred embodiment for treating or preventing the establishment or growth of a fungal infection, the therapeutic agent is an antifungal compound, radionuclide, or oligonucleotide. A therapeutic agent can have a therapeutic effect by slowing down or inhibiting growth of a cell, or by killing or inducing cell death (apoptosis) in a cell.

“Treatment” of a disease refers to improving, curing, or preventing at least one symptom of the disease.

I. Targeting Moieties

The targeting moiety, which assists the construct in localizing to a particular target area, entering a target cell(s), and/or binding to a target receptor, may be selected on the basis of the particular condition or site to be treated or imaged. The targeting moiety may further comprise any of a number of different chemical entities. In one embodiment, the targeting moiety is a small molecule.

Receptor mediated endocytotic activity has been utilized for delivering exogenous molecules such as proteins and nucleic acids to cells. Generally, a specified ligand is chemically conjugated by covalent, ionic, or hydrogen bonding to an exogenous molecule of interest (i.e., the exogenous compound), forming a conjugate molecule having a moiety (the ligand portion) that is still recognized in the conjugate by a target receptor. Using this technique, the phototoxic protein psoralen has been conjugated to insulin and internalized by the insulin receptor endocytotic pathway (Gasparro, Biochem. Biophys. Res. Comm. 141(2), pp. 502-509, Dec. 15, 1986); the hepatocyte-specific receptor for galactose terminal asialoglycoproteins has been utilized for the hepatocyte-specific transmembrane delivery of asialoorosomucoid-poly-L-lysine non-covalently complexed to a DNA plasmid (Wu, G. Y., J. Biol. Chem., 262(10), pp. 4429-4432, 1987); the cell receptor for epidermal growth factor has been utilized to deliver polynucleotides covalently linked to EGF to the cell interior (Myers, European Patent Application 86810614.7, published Jun. 6, 1988); the intestinally situated cellular receptor for the organometallic vitamin B₁₂-intrinsic factor complex has been used to mediate delivery to the circulatory system of a vertebrate host a drug, hormone, bioactive peptide or immunogen complexed with vitamin B₁₂ and delivered to the intestine through oral administration (Russell-Jones et al., European patent Application 86307849.9, published Apr. 29, 1987); the mannose-6-phosphate receptor has been used to deliver low density lipoproteins to cells (Murray, G. J. and Neville, D. M., Jr., J.Bio.Chem, Vol. 255 (24), pp. 1194-11948, 1980); the cholera toxin binding subunit receptor has been used to deliver insulin to cells lacking insulin receptors (Roth and Maddox, J. Cell. Phys. Vol. 115, p. 151, 1983); and the human chorionic gonadotropin receptor has been employed to deliver a ricin a-chain coupled to HCG to cells with the appropriate HCG receptor in order to kill the cells (Oeltmann and Heath, J. Biol. Chem, vol. 254, p. 1028 (1979)).

A particularly preferred embodiment is biotin, a naturally occurring vitamin, which has been shown to localize effectively to tumors and sites of infection. Furthermore, as described in U.S. Pat. No. 5,716,594, imaging agents and therapeutics have been successfully delivered to such sites when coupled to biotin. Another preferred small molecule targeting moiety is folate (see U.S. Pat. No. 5,820,847). Folates are particularly useful in targeting cancer cells, since a variety of carcinomas overexpress folate receptors. See Ladino et al. (Int J Cancer 1997, 73(6):859-6). Riboflavin and its derivatives are other small molecule targeting moieties for targeting delivery of constructs to cancer cells (see, for example, U.S. Pat. No. 5,688,488). Additional nutrients believed to trigger receptor-mediated endocytosis and therefore useful targeting moieties of the instant claims include carnitine, inositol, lipoic acid, niacin, pantothenic acid, thiamin, pyridoxal, ascorbic acid, and the lipid soluble vitamins A, D, E and K. A second exemplary type of small molecule targeting moiety includes steroidal lipids, such as cholesterol, and steroidal hormones, such as estradiol, testosterone, etc.

In another embodiment, the targeting moiety may comprise a protein. Particular types of proteins may be selected based on known characteristics of the target site or target cells. For example, the probe can be an antibody either monoclonal or polyclonal, where a corresponding antigen is displayed at the target site. In situations wherein a certain receptor is expressed by the target cells, the targeting moiety may comprise a protein or peptidomimetic ligand capable of binding to that receptor. Proteins ligands of known cell surface receptors include low density lipoproteins, transferrin, insulin, fibrinolytic enzymes, anti-HER2, platelet binding proteins such as annexins, and biological response modifiers (including interleukin, interferon, erythropoietin and colony-stimulating factor). Also, anti-EGF receptor antibodies, which internalize following binding to the receptor and traffic to the nucleus to an extent, are preferred targeting moieties for use in the present invention to facilitate delivery of Auger emitters and nucleus binding drugs to target cell nuclei.

A number of monoclonal antibodies that bind to a specific type of cell have been developed, including monoclonal antibodies specific for tumor-associated antigens in humans. Among the many such monoclonal antibodies that may be used are anti-TAC, or other interleukin-2 receptor antibodies; 9.2.27 and NR-ML-05 to the 250 kilodalton human melanoma-associated proteoglycan; and NR-LU-10 to a pancarcinoma glycoprotein. An antibody employed in the present invention may be an intact (whole) molecule, a fragment thereof, or a functional equivalent thereof. Examples of antibody fragments are F(ab′)₂, Fab′, Fab, and Fᵥ fragments, which may be produced by conventional methods or by genetic or protein engineering.

Other preferred targeting moieties include sugars (e.g., glucose, fucose, galactose, mannose) that are recognized by target-specific receptors. For example, instant claimed constructs can be glycosylated with mannose residues (e.g., attached as C-glycosides to a free nitrogen) to yield targeted constructs having higher affinity binding to tumors expressing mannose receptors (e.g., glioblastomas and gangliocytomas), and bacteria, which are also known to express mannose receptors (Bertozzi, C R and M D Bednarski Carbohydrate Research 223:243 (1992); J. Am. Chem. Soc. 114:2242,5543 (1992)), as well as potentially other infectious agents. Certain cells, such as malignant cells and blood cells (e.g., A, AB, B, etc.) display particular carbohydrates, for which a corresponding lectin may serve as a targeting moiety.

Additional ligands which may be suitable for use as targeting moieties in the present invention include haptens, epitopes, and dsDNA fragments and analogs and derivatives thereof Such moieties bind specifically to antibodies, fragments or analogs thereof, including mimetics (for haptens and epitopes), and zinc finger proteins (for dsDNA fragments).

TABLE I
Exemplary Targets and Targeting Moieties

| Target | Targeting Moiety |
|---|---|
| Cell-surface receptor | Receptor Ligand |
| Hapten, epitope | Antibody |
| dsDNA fragment | Zinc finger protein |
| Carbohydrate | Lectin |
| Enzyme | Enzyme inhibitor |

TABLE II
Exemplary Tissue-Selective Targeting Moieties

| Cell Type(s) | Targeting Moiety |
|---|---|
| liver cells | galactose |
| Kupffer cells; cancer cells | mannose |
| expressing mannose receptors | |
| (e.g., glioblastomas and ganglic | |
| cytomas) | |
| adipose tissue | insulin |
| lymphocytes | Antibody to CD4, and gp120 |
| fibroblasts | mannose-6-phosphate |
| nerve cells | Apolipoprotein E |
| lung | Antibody to polymeric |
| | immunoglobulin receptor (Pig R) |
| enterocyte | Vitamin B₁₂ |
| prostate cancer cells | antibody to prostate specific antigen |
| | or prostate specific membrane |
| | antigen |
| breast cancer cells | antibody to Her2 antigen |

Antibodies are effective ways of targeting cells that express particular antigens on the cell surface, and thus can be used to selectively target particular cells, such as cancer cells or cells from a particular tissue. Furthermore, antibodies may be made by using standard protocols (See, for example, Antibodies: A Laboratory Manual, ed. Harlow and Lane (Cold Spring Harbor Press: 1988)). A mammal, such as a mouse, a hamster or rabbit can be immunized with an immunogenic form of the peptide (e.g., a polypeptide or an antigenic fragment which is capable of eliciting an antibody response, or a fusion protein as described above).

In one exemplary technique, following immunization of an animal with an antigenic preparation of a polypeptide, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a polypeptide of the present invention and monoclonal antibodies isolated from a culture comprising such hybridoma cells.

The term antibody as used herein is intended to include fragments thereof which are also specifically reactive with one of the subject mammalian polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a subject protein conferred by at least one CDR region of the antibody.

Preferred targeting moieties facilitate binding of the construct to their respective target molecules with an affinity of at least about k_{D} 10-6 M, preferably 10⁻⁷ M, more preferably 10⁻⁸ M, and most preferably 10⁻⁹ M. Binding of the targeting moiety to its receptor should be sufficient to allow a significant amount of the targeting moiety to bind sufficiently long to allow the targeting moiety to be taken into the cell. The affinity of a ligand for a receptor can be determined according to methods well known in the art.

Preferred targeted constructs exhibit a high target to non-target ratio when administered in vivo. Preferably the ratio is at least of 2:1, even more preferably at least 3:1; and most preferably at least 5:1 (i.e., it is 2, 3, or 5 times more likely that the target moiety will bind to its specific receptor, relative to other receptors). In certain embodiments, the targeting construct will be administered locally to a subject, or to specific cells in vitro. In such embodiments, it may not be of consequence that the targeting moiety may also interact with cell surface molecules that are located on different tissues, since the targeting construct will not reach that site. Thus, the level of specificity of the targeting moiety depends on factors, such as the type of administration of the targeting construct.

The reactivity of a targeting moiety towards structures other than the targeted receptor can be determined by assays, e.g., by labeling the targeting moiety or using a labeled targeting construct; incubating it with tissue slices; and determining the location of the label. Assays can also be done in animals, such as mice or rats. For example, a targeting construct can be administered to a non-human animal and the amount of construct present at various locations is determined, e.g., as described in the Examples.

In certain embodiments, the targeting construct may comprise an internalizing polypeptide sequence, such as antepennepedia protein, HIV transactivating (Tat) protein, mastoparan (T. Higashijima et al. (1990) J. Biol. Chem. 265:14176), melittin, bombolittin, delta hemolysin, pardaxin, Pseudomonas exotoxin A, clathrin, Diphtheria toxin, C9 complement protein, or a fragment of one of the preceding proteins. An internalizing peptide is capable of crossing a cellular membrane by, e.g., transcytosis, at a relatively high rate, and thereby promote cellular uptake of molecules to which they are attached. Certain internalizing polypeptides, such as Tat, are also known to localize to the nucleus or other cellular structures. Thus a targeted construct of the present invention which includes such an internalizing peptide sequence may exhibit increased uptake by target cells relative to constructs that lack such a sequence.

The internalizing polypeptide may be part of the targeting moiety or a separate element of the targeting construct. In one embodiment of the invention, the internalizing polypeptide serves as the targeting moiety (see examples below of such targeting moieties). In another embodiment, the internalizing polypeptide is covalently linked to one ore more of the other elements of the targeting construct. For example, the internalizing polypeptide can be linked to the targeting moiety; to the nucleic acid; to the payload; to the targeting moiety and to the nucleic acid; or to the targeting moiety and the payload. The preferred location of an internalizing polypeptide in a targeting construct can be determined, e.g., by conduction in vitro assays using target cells, labeled targeting construct, and determining the amount of label that is incorporated into the cells.

In one embodiment, the internalizing peptide is derived from the drosophila antepennepedia protein, or homologs thereof. The 60 amino acid long homeodomain of the homeo-protein antepennepedia has been demonstrated to translocate through biological membranes and can facilitate the translocation of heterologous polypeptides to which it is couples. See for example Derossi et al. (1994) J Biol Chem 269:10444-10450; Perez et al. (1992) J Cell Sci 102:717-722. Recently, it has been demonstrated that fragments as small as 16 amino acids long of this protein are sufficient to drive internalization. See Derossi et al. (1996) J Biol Chem 271:18188-18-193. The present invention contemplates a targeting construct comprising at least a portion of the antepennepedia protein (or homolog thereof) sufficient to increase the transmembrane transport of the targeting construct, relative to the targeting construct alone, by a statistically significant amount.

Another example of an internalizing peptide is the HIV transactivator (TAT) protein. This protein appears to be divided into four domains (Kuppuswamy et al. (1989) Nucl. Acids Res. 17:3551-3561). Purified TAT protein is taken up by cells in tissue culture (Frankel, et al. (1989) Cell 55:1189-1193), and peptides, such as the fragment corresponding to residues 37-62 of TAT, are rapidly taken up by cell in vitro (Green, et al. (1989) Cell 55:1179-1188). The highly basic region mediates internalization and targeting of the internalizing moiety to the nucleus (Ruben et al. (1989) J. Virol. 63:1-8). Peptides or analogs that include a sequence present in the highly basic region, such as CFITKALGISYGRKKRRQRRRPPQGS (SEQ ID NO: 1), are conjugated to a targeting construct to aid in internalization and targeting those constructs to the intracellular milieu.

While not wishing to be bound by any particular theory, it is noted that hydrophilic polypeptides may be also be physiologically transported across the membrane barriers by coupling or conjugating a targeting construct to a transportable peptide which is capable of crossing the membrane by receptor-mediated transcytosis. Suitable internalizing peptides of this type can be generated using all or a portion of, e.g., a histone, insulin, transferrin, basic albumin, prolactin and insulin-like growth factor I (IGF-I), insulin-like growth factor II (IGF-II) or other growth factors. For instance, it has been found that an insulin fragment, showing affinity for the insulin receptor on capillary cells, and being less effective than insulin in blood sugar reduction, is capable of transmembrane transport by receptor-mediated transcytosis. Preferred growth factor-derived internalizing peptides include EGF (epidermal growth factor)-derived peptides, such as CMHIESLDSYTC (SEQ ID NO: 2) and CMYIEALDKYAC (SEQ ID NO: 3); TGF-beta (transforming growth factor beta)-derived peptides; peptides derived from PDGF (platelet-derived growth factor) or PDGF-2; peptides derived from IGF-I (insulin-like growth factor) or IGF-II; and FGF (fibroblast growth factor)-derived peptides. Hydrophilic polypeptides can be included in a targeting construct, or they can constitute the targeting moiety.

Another class of translocating/internalizing peptides exhibits pH-dependent membrane binding. For an internalizing peptide that assumes a helical conformation at an acidic pH, the internalizing peptide acquires the property of amphiphilicity, e.g., it has both hydrophobic and hydrophilic interfaces. More specifically, within a pH range of approximately 5.0-5.5, an internalizing peptide forms an alpha-helical, amphiphilic structure that facilitates insertion of the moiety into a target membrane. An alpha-helix-inducing acidic pH environment may be found, for example, in the low pH environment present within cellular endosomes. Such internalizing peptides can be used to facilitate transport of targeting cosntructs, taken up by an endocytic mechanism, from endosomal compartments to the cytoplasm.

A preferred pH-dependent membrane-binding internalizing peptide includes a high percentage of helix-forming residues, such as glutamate, methionine, alanine and leucine. In addition, a preferred internalizing peptide sequence includes ionizable residues having pKa's within the range of pH 5-7, so that a sufficient uncharged membrane-binding domain will be present within the peptide at pH 5 to allow insertion into the target cell membrane.

A particularly preferred pH-dependent membrane-binding internalizing peptide in this regard is aa1-aa2-aa3-EAALA(EALA)4-EALEALAA-amide (SEQ ID NO: 4), which represents a modification of the peptide sequence of Subbarao et al. (Biochemistry 26:2964 (1987)). Within this peptide sequence, the first amino acid residue (aa1) is preferably a unique residue, such as cysteine or lysine, that facilitates chemical conjugation of the internalizing peptide to a targeting protein conjugate. Amino acid residues 2-3 may be selected to modulate the affinity of the internalizing peptide for different membranes. For instance, if both residues 2 and 3 are lys or arg, the internalizing peptide will have the capacity to bind to membranes or patches of lipids having a negative surface charge. If residues 2-3 are neutral amino acids, the internalizing peptide will insert into neutral membranes.

Yet other preferred internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, exemplary internalizing peptides may be modified through attachment of substituents that enhance the alpha-helical character of the internalizing peptide at acidic pH.

Yet another class of internalizing peptides suitable for use within the present invention include hydrophobic domains that are “hidden” at physiological pH, but are exposed in the low pH environment of the target cell endosome. Upon pH-induced unfolding and exposure of the hydrophobic domain, the moiety binds to lipid bilayers and effects translocation of a covalently linked targeting construct into the cell cytoplasm. Such internalizing peptides may be modeled after sequences identified in, e.g., Pseudomonas exotoxin A, clathrin, or Diphtheria toxin.

Pore-forming proteins or peptides may also serve as internalizing peptides herein. Pore forming proteins or peptides may be obtained or derived from, for example, C9 complement protein, cytolytic T-cell molecules or NK-cell molecules. These moieties are capable of forming ring-like structures in membranes, thereby allowing transport of attached targeting construct through the membrane and into the cell interior.

Mere membrane intercalation of an internalizing peptide may be sufficient for translocation of a targeting construct across cell membranes. However, translocation may be improved by attaching to the internalizing peptide a substrate for intracellular enzymes (i.e., an “accessory peptide”). It is preferred that an accessory peptide be attached to a portion(s) of the internalizing peptide that protrudes through the cell membrane to the cytoplasmic face. The accessory peptide may be advantageously attached to one terminus of a translocating/internalizing moiety or anchoring peptide. An accessory moiety of the present invention may contain one or more amino acid residues. In one embodiment, an accessory moiety may provide a substrate for cellular phosphorylation (for instance, the accessory peptide may contain a tyrosine residue).

An exemplary accessory moiety in this regard would be a peptide substrate for N-myristoyl transferase, such as GNAAAARR (SEQ ID NO: 5) (Eubanks et al. (1988) Peptides. Chemistry and Biology, Garland Marshall (ed.), ESCOM, Leiden 566-69). In this construct, an internalizing, peptide would be attached to the C-terminus of the accessory peptide, since the N-terminal glycine is critical for the accessory moiety's activity. This hybrid peptide, upon attachment to a targeting cosntruct is N-myristylated and will be translocated across the cell membrane.

II. Nucleic Acids

The oligonucleotide portion of the subject targeted constructs, designed to be complementary to a nucleic acid of interest, may inhibit the transcription of a related gene, serve as a probe for the expression of that gene, assist in localizing the construct in the cell, promote retention of the construct by the target cell, or any combination thereof. In preferred embodiments, the nucleic acid portion of the subject constructs serves to augment the targeting moiety by selectively promoting retention of the construct by target cells which express a particular nucleic acid.

Oligonucleotide portions of the invention may comprise any polymeric compound capable of specifically binding to a target polynucleotide by way of a regular pattern of monomer-to-nucleoside interactions, such as Watson-Crick type of base pairing, Hoogsteen or reverse Hoogsteen types of base pairing, or the like. The oligonucleotide portion may be modified to enhance its efficacy, pharmacokinetic properties, or physical properties. For example, it is known that enhanced lipid solubility and/or resistance to nuclease digestion results by substituting an alkyl group or alkoxy group for a phosphate oxygen in the internucleotide phosphodiester linkage to form an alkylphosphonate oligonucleoside or alkylphosphotriester oligonucleotide. Non-ionic oligonucleotides such as these are characterized by increased resistance to nuclease hydrolysis and/or increased cellular uptake, while retaining the ability to form stable complexes with complementary nucleic acid sequences. The alkylphosphonates, in particular, are stable to nuclease cleavage and soluble in lipid. The preparation of alkylphosphonate oligonucleosides is disclosed in Tso et al., U.S. Pat. No. 4,469,863.

Preferably, nuclease resistance is conferred on the constructs of the invention by providing nuclease-resistant internucleosidic linkages. Many such linkages are known in the art, e.g., phosphorothioate: Zon and Geiser, Anti-Cancer Drug Design, 6:539-568 (1991); Stec et al., U.S. Pat. No. 5,151,510; Hirschbein, U.S. Pat. No. 5,166,387; Bergot, U.S. Pat. No. 5,183,885; phosphorodithioates: Marshall et al., Science, 259:1564-1570 (1993); Caruthers and Nielsen, International application PCT/US89/02293; phosphoramidates, e.g., —OP(═O)(NR₁ R₂)—O— with R₁ and R₂ hydrogen or C₁-C₃ alkyl; Jager et al., Biochemistry, 27:7237-7246 (1988); Froehler et al., International application PCT/US90/03138; peptide nucleic acids: Nielsen et al., Anti-Cancer Drug Design, 8:53-63 (1993), International application PCT/EP92/01220; methylphosphonates: Miller et al., U.S. Pat. No. 4,507,433, Ts'o et al., U.S. Pat. No. 4,469,863; Miller et al., U.S. Pat. No. 4,757,055; and P-chiral linkages of various types, especially phosphorothioates, Stec et al., European patent application 506,242 (1992) and Lesnikowski, Bioorganic Chemistry, 21:127-155 (1993). Additional nuclease-resistant linkages include phosphoroselenoate, phosphorodiselenoate, phosphoroanilothioate, phosphoranilidate, alkylphosphotriester such as methyl- and ethylphosphotriester, carbonates such as carboxymethyl ester, carbamate, morpholino carbamate, 3′-thioformacetal, silyl such as dialkyl (C₁-C₆)- or diphenylsilyl, sulfamate ester, and the like. Such linkages and methods for introducing them into oligonucleotides are described in many references, e.g., reviewed generally by Peyman and Ulmann, Chemical Reviews 90:543-584 (1990); Milligan et al., J. Med. Chem., 36:1923-1937 (1993); Matteucci et al., International application PCT/US91/06855.

Resistance to nuclease digestion may also be achieved by modifying the internucleotide linkage at both the 5′ and 3′ termini with phosphoroamidites according to the procedure of Dagle et al., Nucl. Acids Res. 18, 4751-4757 (1990).

Preferably, phosphorus analogs of the phosphodiester linkage are employed in the compounds of the invention, such as phosphorothioate, phosphorodithioate, phosphoramidate, or methylphosphonate. More preferably, phosphorothioate is employed as the nuclease resistant linkage.

Phosphorothioate oligonucleotides contain a sulfur-for-oxygen substitution in the internucleotide phosphodiester bond. Phosphorothioate oligonucleotides combine the properties of effective hybridization for duplex formation with substantial nuclease resistance, while retaining the water solubility of a charged phosphate analogue. The charge is believed to confer the property of cellular uptake via a receptor (Loke et al., Proc. Natl. Acad. Sci., 86, 3474-3478 (1989)).

It is understood that in addition to the preferred linkage groups, compounds of the invention may comprise additional modifications, e.g., boronated bases, Spielvogel et al., 5,130,302; cholesterol moieties, Shea et al., Nucleic Acids Research, 18:3777-3783 (1990) or Letsinger et al., Proc. Natl. Acad. Sci., 86:6553-6556 (1989); and 5-propynyl modification of pyrimidines, Froehler et al., Tetrahedron Lett., 33:5307-5310 (1992).

Preferably, oligonucleotide portions of the invention are synthesized by conventional means on commercially available automated DNA synthesizers, e.g., an Applied Biosystems (Foster City, Calif.) model 380B, 392 or 394 DNA/RNA synthesizer. Preferably, phosphoramidite chemistry is employed, e.g., as disclosed in the following references: Beaucage and Iyer, Tetrahedron, 48:2223-2311 (1992); Molko et al., U.S. Pat. No. 4,980,460; Koster et al., U.S. Pat. No. 4,725,677; Caruthers et al., U.S. Pat. Nos. 4,415,732; 4,458,066; and 4,973,679.

In embodiments where triplex formation is desired, there are constraints on the selection of target sequences. Generally, third strand association via Hoogsteen type of binding is most stable along homopyrimidine-homopurine tracks in a double stranded target. Usually, base triplets form in T-A*T or C-G*C motifs (where “-” indicates Watson-Crick pairing and “*” indicates Hoogsteen type of binding); however, other motifs are also possible. For example, Hoogsteen base pairing permits parallel and antiparallel orientations between the third strand (the Hoogsteen strand) and the purine-rich strand of the duplex to which the third strand binds, depending on conditions and the composition of the strands. There is extensive guidance in the literature for selecting appropriate sequences, orientation, conditions, nucleoside type (e.g., whether ribose or deoxyribose nucleosides are employed), base modifications (e.g., methylated cytosine, and the like) in order to maximize, or otherwise regulate, triplex stability as desired in particular embodiments, e.g., Roberts et al., Proc. Natl. Acad. Sci., 88:9397-9401 (1991); Roberts et al., Science, 58:1463-1466 (1992); Distefano et al., Proc. Natl. Acad. Sci., 90:1179-1183 (1993); Mergny et al., Biochemistry, 30:9791-9798 (1992); Cheng et al., J. Am. Chem. Soc., 114:4465-4474 (1992); Beal and Dervan, Nucleic Acids Research, 20:2773-2776 (1992); Beal and Dervan, J. Am. Chem. Soc., 114:4976-4982; Giovannangeli et al., Proc. Natl. Acad. Sci., 89:8631-8635 (1992); Moser and Dervan, Science, 238:645-650 (1987); McShan et al., J. Biol. Chem., 267:5712-5721 (1992); Yoon et al., Proc. Natl. Acad. Sci., 89:3840-3844 (1992); and Blume et al., Nucleic Acids Research, 20:1777-1784 (1992).

The length of the oligonucleotide moieties may be sufficiently large to ensure that specific binding will take place only at the desired target polynucleotide and not at other adventitious sites, as explained in many references, e.g., Rosenberg et al., International application PCT/US92/05305; or Szostak et al., Meth. Enzymol, 68:419-429 (1979). The desired length is determined by several factors, including the inconvenience and expense of synthesizing and purifying oligomers greater than about 30-40 nucleotides in length, the greater tolerance of longer oligonucleotides for mismatches than shorter oligonucleotides, whether modifications to enhance binding or specificity are present, whether duplex or triplex binding is desired, and the like. Usually, oligonucleotides useful in the invention have lengths in the range of about 12 to 60 nucleotides. More preferably, compounds of the invention have lengths in the range of about 15 to 40 nucleotides; and most preferably, they have lengths in the range of about 18 to 30 nucleotides.

In general, the oligonucleotides used in the practice of the present invention will have a sequence which is completely complementary to a selected portion of the target polynucleotide. Absolute complementarity is not however required, particularly in larger oligomers. Thus, reference herein to a “nucleotide sequence complementary to” a target polynucleotide does not necessarily mean a sequence having 100% complementarity with the target segment. In general, any oligonucleotide having sufficient complementarity to form a stable duplex with the target (e.g., an oncogene mRNA), that is, an oligonucleotide which is “hybridizable”, is suitable. Stable duplex formation depends on the sequence and length of the hybridizing oligonucleotide and the degree of complementarity with the target polynucleotide. Generally, the larger the hybridizing oligomer, the more mismatches may be tolerated. More than one mismatch may not be suitable for oligomers of less than about 21 nucleotides. One skilled in the art may readily determine the degree of mismatching which may be tolerated between any given oligomer and the target sequence, based upon the melting point, and therefore the thermal stability, of the resulting duplex.

The thermal stability of hybrids formed by the oligonucleotides of the invention may be determined by way of melting, or strand dissociation, curves. The temperature of fifty percent strand dissociation is taken as the melting temperature, Tₘ, which, in turn, provides a convenient measure of stability. Tₘ measurements are typically carried out in a saline solution at neutral pH with target and oligonucleotide concentrations at between about 1.0-2.0 M. Typical conditions are as follows: 150 mM NaCl and 10 mM MgCl₂ in a 10 mM sodium phosphate buffer (pH 7.0) or in a 10 mM Tris-HCl buffer (pH 7.0). Data for melting curves are accumulated by heating a sample of the oligonucleotide/target polynucleotide complex from room temperature to about 85 C. As the temperature of the sample increases, absorbance of 260 nm light is monitored at 1 C. intervals, e.g., using a Cary (Australia) model 1E or a Hewlett-Packard (Palo Alto, Calif.) model HP 8459 UV/VIS spectrophotometer and model HP 89100A temperature controller, or like instruments. Such techniques provide a convenient means for measuring and comparing the binding strengths of oligonucleotides of different lengths and compositions.

In certain embodiments, the nucleic acid portion may function to inhibit or suppress the transcription of a gene by functioning as an antisense oligonucleotide.

Where the target polynucleotide comprises an mRNA transcript, oligonucleotides complementary to and hybridizable with any portion of the transcript are, in principle, effective for inhibiting translation. This occurs because each protein synthesized by a cell is encoded by a specific messenger mRNA (mRNA). If translation of a specific RNA is inhibited, the protein product derived from this translation will likewise be reduced. Oligonucleotide sequences designed to be complementary (antisense) to a specific target mRNA sequence will bind to the target sequence thereby inhibiting translation of that specific mRNA. It is believed that an antisense oligonucleotide, by hybridizing to the RNA and forming a complex, blocks target mRNA ribosomal binding causing translational inhibition. Alternatively, the duplex that is formed by the sense and antisense molecules may be easier to degrade. Other theories describe complexes that antisense RNA could form with complementary DNA to inhibit mRNA transcription. Thus, an antisense oligonucleotide might inhibit the translation of a given gene product by either directly inhibiting translation or through inhibition of transcription.

It is believed that translation is most effectively inhibited by blocking the mRNA at a site at or near the initiation codon. Thus, oligonucleotides complementary to the 5′-region of mRNA transcript are preferred. Oligonucleotides complementary to the target mRNA, including the initiation codon (the first codon at the 5′ end of the translated portion of the transcript), or codons adjacent the initiation codon, are preferred.

While antisense oligomers complementary to the 5′-region of the target mRNA transcripts are preferred, particularly the region including the initiation codon, it should be appreciated that useful antisense oligomers are not limited to those oligomers complementary to the sequences found in the translated portion of the mRNA transcript, but also includes oligomers complementary to nucleotide sequences contained in, or extending into, the 5′- and 3′-untranslated regions, as well as in the promoter region and introns. In certain embodiments, a targeting construct includes a “sense” nucleic acid.

Also within the scope of the invention are targeting constructs comprising two or more nucleic acid molecules. The nucleic acids can be directed to the same gene, or alternatively, they can be directed to (or complementary to) different genes. For example, if the target cell is a cell expressing high levels of c-myb RNA and c-fos RNA, the targeting construct may include an oligonucleotide that is complementary to c-myb RNA and an oligonucleotide that is complementary to c-fos RNA. The different nucleic acids may be covalently linked to each other, or they can not be linked to each other.

Nucleic acids of targeting construct are preferably single stranded. The nucleic acids are preferably from about 12 to about 100 nucleotides, more preferably from about 12 to about 50 nucleotides long, and even more preferably from about 15 to about 25 nucleotides long. However, nucleic acids having from about 100 to about 200, 500 or 1000 nucleotides are also within the scope of the invention. Larger nucleotides can also be employed according to the invention. The term “oligonucleotide” as used herein is used interchangeably herein with single stranded nucleic acid, and is not intended to be limited in the number of nucleotides.

Exemplary target nucleic acids include those that are expressed at high levels in target cells. For example, when targeting cancer cells, the target nucleic acid may be complementary to RNA of an oncogene, e.g., c-myc, c-ras, c-fos, or c-jun. The potential for clinical development of antisense inhibitors of ras is discussed, e.g., in by Cowsert, L. M., Anti-Cancer Drug Design (1997) 12:359-371.

Generally, the following protocol may be followed when choosing a nucleic acid to incorporate into a targeting construct for targeting a particular cell. One or more genes expressed at high levels in the target cell are identified. Such genes may be known from the literature, or alternatively, they can be identified. For example, RNA from the cell line can be extracted, cDNA synthesized from the RNA, and the cDNA hybridized to a blot or an array comprising the DNA of various genes. One or more target genes can then be selected based on the hybridization results. Preferred target genes are those that are not significantly expressed in other cell types, at least in cells that are close to the targeting cells. Thus, house-keeping genes might not be the best choice for certain embodiments. Antisense nucleic acids that are complementary to different portions of one or more potential target genes can then be prepared, e.g., by PCT amplification, or synthetically. These nucleic acids can then be incorporated into a targeting construct, and the level of incorporation and retention of the targeting construct can be determined, e.g., as described in the Examples.

III. Payloads

The targeted constructs of the present invention may include any of a wide variety of chemical entities to be delivered to the target site or into target cells. Generally, the payloads may be categorized as imaging agents and therapeutic agents. Imaging agents comprise those payloads which are detectable, e.g., by emitting light, radioactive emissions, or chemical signals, by absorbing radiation (e.g., x-rays), or by otherwise changing a characteristic of treated cells relative to untreated cells. Therapeutic agents include payloads which are biologically active, preferably by countering the abnormal condition of the targeted site (e.g., tumor or infection).

A therapeutic agent useful in a targeted construct may be any of a number of chemical entities, e.g., an enzyme, drug, radionuclide, enzyme inhibitor, etc. For example, moieties useful as therapeutic agents include amino acids and their derivatives; analgesics such as acetaminophen, aspirin, and ibuprofen; antiasthmatics; anticonvulsants; antidepressants such as amitriptyline, fluoxetine, nortriptyline, and imipramine; antiemetics; antifungal agents including: allyamines, imidazoles, polyenes, and triazoles; antigens and antibodies thereto; antihistamines such as chlorpheniramine and brompheniramine; antihypertensive agents such as clonidine, methyldopa, prazosin, verapamil, nifedipine, captopril, and enalapril; antiinflammatory agents including non-steroidal agents, such as aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives thiazinecarboxamides and others, as well as steroidal agents, such as glucocorticoids; antimicrobials such as aminoglycosides, amphenicols, cinoxacin, ciprofloxacin, 2,4-diaminopyrimidines, -lactams (e.g. carbapenems, cephalosporins, cephamycins, monobactams, oxacephems and penicillins), lincosamides, macrolides, nitrofurans, norfloxacin, peptides, polypeptides, and proteins (e.g. defensins, bacitracin, polymyxin, cecropins, magainin II, indolicidin, ranalexin, protegrins, gallinacins, tritrpticin, lactoferricin, drosomycin, holotricin, thanatin, dermaseptin, iturins, syringomycins, nikkomycins, polyoxins, FR-900403, echinocandins, pneumocandins, aculeacins, mulundocandins, WF11899, aureobasidins, schizotrin A, cepacidines, zeamatin, cyclopeptides and D4el), quinolones and analogs, sulfonamides, sulfones, tetracyclines; antinauseants; anti-Parkinson agents; antispasmodics; apoproteins, bronchodilators such as albuterol and theophylline; antivirals including: purines/pyrimidinones (e.g. acyclovir, dideoxy-cytidine, -adenosine, or -inosine, interferons, amantadine, ribavirin); beta-blockers such as propranolol, metoprolol, atenolol, labetolol, timolol, penbutolol, and pindolol; cancer drugs including chemotherapeutic agents; cardiovascular agents including antiarrhythmics, cardiac glycosides, antianginals and vasodilators; central nervous system agents including stimulants, psychotropics, antimanics, and depressants; coenzymes; cough suppressants; decongestants; diuretics; enzymes; enzyme inhibitors; expectorants; glycoproteins; H-2 antagonists such as nizatidine, cimetidine, famotidine, and ranitidine; haptens and antibodies thereto; hormones, lipids, liposomes; mucolytics; muscle relaxants; protein analogs in which at least one non-peptide linkage replaces a peptide linkage; phospholipids; prostaglandins; radionuclides (e.g. ¹³¹I, ¹⁸⁶Re, ¹⁸⁸Re, ⁹⁰Y, ²¹²Bi, ²¹¹At, ⁸⁹Sr, ¹⁶⁶Ho, ¹⁵³Sm, ⁶⁷Cu and ⁶⁴Cu; receptors and other membrane proteins; retro-inverso oligopeptides; stimulants; toxins such as aflatoxin, digoxin, rubratoxin, and xanthotoxin; tranquilizers such as diazepam, chordiazepoxide, oxazepam, alprazolam, and triazolam; and vitamins and mineral and nutritional additives. For other therapeutic agents, see, e.g., the Merck Index. In addition to therapeutic agents that are currently in use, the instant invention contemplates agents that are in development or will be developed and that are useful for treating or preventing the progression of an infection, inflammatory response, tumor, or other abnormal condition.

Targeted constructs can alternatively or additionally be labeled with any of a variety of imaging agents which are known in the art and which will depend to some extent on the means used to detect or monitor the compound in vivo or in vitro. Preferred imaging agents for performing positron emission tomography (PET) and single photon emission computer tomography (SPECT) include F-18, Tc-99m, and 1-123. Preferred imaging agents for magnetic resonance imaging (MRI) include an appropriate atom with unpaired spin electrons or a free radical.

When the payload is intended to perform in an imaging capacity, the payload comprises a moiety such as a radionuclide or paramagnetic contrast agent, fluorescent or chemiluminescent label, or other type of detectable marker. The imaging agents described above may contain any label in accordance with the invention. Highly specific and sensitive labels are provided by radionuclides, which can then be detected using positron emission tomography (PET) or Single Photon Emission Computed Tomography (SPECT) imaging. More preferably, the imaging agent of the invention contains a radionuclide selected from the group consisting of ¹³¹I, ¹²⁵I, ¹²³I ^{99m}Tc, ¹⁸F, ⁶⁸Ga, ⁶⁷Ga, ⁷²As, ⁸⁹Zr, ⁶⁴Cu, ⁶²Cu, ¹¹¹In, ²⁰³Pb, ¹⁹⁸Hg, ¹¹C, ⁹⁷Ru, and ²⁰¹Tl or a paramagnetic contrast agent, such as gadolinium, cobalt, nickel, manganese, and iron. As will be discussed below, these atoms may be directly incorporated into the targeting moiety or the oligonucleotide, or may be attached through a separate chemical structure. Additional information relating to the use of chelated radionuclides may be found in U.S. Pat. No. 5,783,171 and 5,688,488.

IV. Method for Making Targeted Constructs

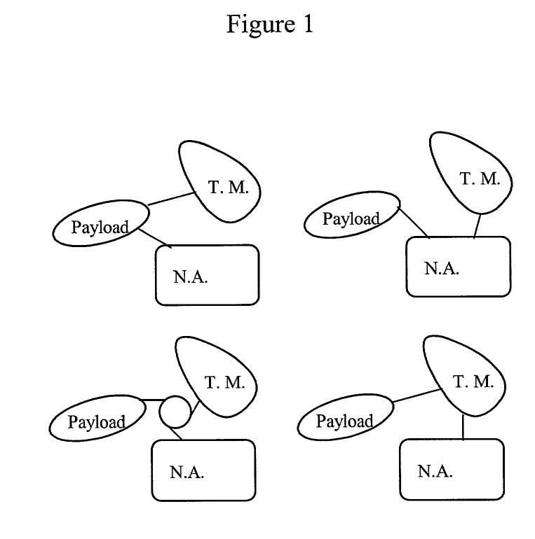

The joining of a targeting moiety, a nucleic acid, and a payload may be effected by any means which produces a link between two or more constituents that is sufficiently stable to withstand the conditions used and which does not alter the function of either constituent. Preferably, the link between them is covalent. The various portions may be assembled in any order or in any configuration that maintains the desired activity of each portion. Two portions may be attached together by linking functional groups present at the termini of those portions or by linking appropriate functional groups present at any location on either portion. Alternatively, all three portions may be joined to a common tether molecule. Such structures are schematically depicted in FIG. 1. Suitable methods for linking the various portions are discussed below.

Numerous chemical cross-linking methods are known and potentially applicable for conjugating the various portions of the instant constructs. Many known chemical cross-linking methods are non-specific, i.e., they do not direct the point of coupling to any particular site on the polypeptide, polynucleotide, or other molecule. As a result, use of non-specific cross-linking agents may attack functional sites or sterically block active sites, rendering the conjugated proteins biologically inactive.

A preferred approach to increasing coupling specificity in the practice of this invention is direct chemical coupling to a functional group found only once or a few times in one or both of the molecules to be cross-linked. For example, in many proteins, cysteine, which is the only protein amino acid containing a thiol group, occurs only a few times. Also, for example, if a polypeptide contains no lysine residues, a cross-linking reagent specific for primary amines will be selective for the amino terminus of that polypeptide. Successful utilization of this approach to increase coupling specificity requires that the molecule have the suitable reactive residues in areas of the molecule that may be altered without loss of the molecule's biological activity.

As demonstrated in the examples below, cysteine residues may be replaced when they occur in parts of a polypeptide sequence where their participation in a cross-linking reaction would likely interfere with biological activity. When a cysteine residue is replaced, it is typically desirable to minimize resulting changes in polypeptide folding. Changes in polypeptide folding are minimized when the replacement is chemically and sterically similar to cysteine. For these reasons, serine is preferred as a replacement for cysteine. As demonstrated in the examples below, a cysteine residue may be introduced into a polypeptide's amino acid sequence for cross-linking purposes. When a cysteine residue is introduced, introduction at or near the amino or carboxy terminus is preferred. Conventional methods are available for such amino acid sequence modifications, whether the polypeptide of interest is produced by chemical synthesis or expression of recombinant DNA.

Coupling of the two constituents can be accomplished via a coupling or conjugating agent. There are several intermolecular cross-linking reagents which can be utilized (see, for example, Means, G. E. and Feeney, R. E., Chemical Modification of Proteins, Holden-Day, 1974, pp. 39-43). Among these reagents are, for example, J-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other cross-linking reagents useful for this purpose include: p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

Cross-linking reagents may be homobifunctional, i.e., having two functional groups that undergo the same reaction. A preferred homobifunctional cross-linking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of polypeptides that contain cysteine residues.

Cross-linking reagents may also be heterobifunctional. Heterobifunctional cross-linking agents have two different functional groups, for example an amine-reactive group and a thiol-reactive group, that will cross-link two proteins having free amines and thiols, respectively. Heterobifunctional cross-linkers provide the ability to design more specific coupling methods for conjugating two chemical entities, thereby reducing the occurrences of unwanted side reactions such as homo-protein polymers. A wide variety of heterobifunctional cross-linkers are known in the art. Examples of heterobifunctional cross-linking agents are succinimidyl 4-(N-maleimidomethyl)-cyclohexane-1-carboxylate (SMCC), N-succinimidyl (4-iodoacetyl) aminobenzoate (SIAB), 1-ethyl-3-(3-dimethylaminopropyl) carbodiimide hydrochloride (EDC); 4-succinimidyloxycarbonyl- a-methyl-a-(2-pyridyldithio)-tolune (SMPT), N-succinimidyl 3-(2-pyridyldithio) propionate (SPDP), succinimidyl 6-[3-(2-pyridyldithio) propionate] hexanoate (LC-SPDP)succinimidyl 4-(N-maleimidomethyl)-cyclohexane-1-carboxylate (“SMCC”), m-maleimidobenzoyl-N-hydroxysuccinimide ester (“MBS”), and succinimide 4-(p-maleimidophenyl)butyrate (“SMPB”), an extended chain analog of MBS. The succinimidyl group of these cross-linkers reacts with a primary amine, and the thiol-reactive maleimide forms a covalent bond with the thiol of a cysteine residue.

Cross-linking reagents often have low solubility in water. A hydrophilic moiety, such as a sulfonate group, may be added to the cross-linking reagent to improve its water solubility. Sulfo-MBS and sulfo-SMCC are examples of cross-linking reagents modified for water solubility.

Another reactive group useful as part of a heterobifunctional cross-linker is a thiol reactive group. Common thiol-reactive groups include maleimides, halogens, and pyridyl disulfides. Maleimides react specifically with free sulfhydryls (cysteine residues) in minutes, under slightly acidic to neutral (pH 6.5-7.5) conditions. Haloalkyl groups (e.g., iodoacetyl functions) react with thiol groups at physiological pH's. Both of these reactive groups result in the formation of stable thioether bonds.

In addition to the heterobifunctional cross-linkers, there exist a number of other cross-linking agents including homobifunctional and photoreactive cross-linkers. Disuccinimidyl -suberate (DSS), bismaleimidohexane (BMH) and dimethylpimelimidate-2 HCl (DMP) are examples of useful homobifunctional cross-linking agents, and bis-[β-(4-azidosalicylamido)ethyl]disulfide (BASED) and N-succinimidyl-6(4′-azido-2′-nitrophenyl-amino)hexanoate (SANPAH) are examples of useful photoreactive cross-linkers for use in this invention. For a recent review of protein coupling techniques, see Means et al. (1990) Bioconjugate Chemistry 1:2-12, incorporated by reference herein.

Many cross-linking reagents yield a conjugate that is essentially non-clearable under cellular conditions. However, some cross-linking reagents contain a covalent bond, such as a disulfide, that is clearable under cellular conditions. For example, dithiobis(succinimidylpropionate) (“DSP”), Traut's reagent and N-succinimidyl 3-(2-pyridyldithio) propionate (“SPDP”) are well-known cleavable cross-linkers. The use of a clearable cross-linking reagent may permit the payload to separate from the construct after delivery to the target. Direct disulfide linkage may also be useful. Additional cleavable linkages are known in the art and may be employed to advantage in certain embodiments of the present invention.

Many methods for linking compounds, such as proteins, labels, and other chemical entities, to nucleotides are known in the art. Some new cross-linking reagents such as n-maleimidobutyryloxy-succinimide ester (“GMBS”) and sulfo-GMBS, have reduced immunogenicity. Substituents have been attached to the 5′ end of preconstructed oligonucleotides using amidite or H-phosphonate chemistry, as described by Ogilvie, K. K., et al., Pure and Appl Chem (1987) 59:325, and by Froehler, B. C., Nucleic Acids Res (1986) 14:5399. Substituents have also been attached to the 3′ end of oligomers, as described by Asseline, U., et al., Tet Lett (1989) 30:2521. This last method utilizes 2,2′-dithioethanol attached to a solid support to displace diisopropylamine from a 3′ phosphonate bearing the acridine moiety and is subsequently deleted after oxidation of the phosphorus. Other substituents have been bound to the 3′ end of oligomers by alternate methods, including polylysine (Bayard, B., et al., Biochemistry (1986) 25:3730; Lemaitre, M., et al., Nucleosides and Nucleotides (1987) 6:311) and, in addition, disulfides have been used to attach various groups to the 3′ terminus, as described by Zuckerman, R., et al., Nucleic Acids Res (1987) 15:5305. It is known that oligonucleotides which are substituted at the 3′ end show increased stability and increased resistance to degradation by exonucleases (Lancelot, G., et al., Biochemistry (1985) 24:2521; Asseline, U., et al., Proc Natl Acad Sci USA (1984) 81:3297). Additional methods of attaching non-nucleotide entities to oligonucleotides are discussed in U.S. Pat. Nos. 5,321,131 and 5,414,077.

Alternatively, an oligonucleotide may include one or more modified nucleotides having a group attached via a linker arm to the base. For example, Langer et al (Proc. Natl. Acad. Sci. U.S.A., 78(11):6633-6637, 1981) describes the attachment of biotin to the C-5 position of dUTP by an allylamine linker arm. The attachment of biotin and other groups to the 5-position of pyrimidines via a linker arm is also discussed in U.S. Pat. No. 4,711,955. Nucleotides labeled via a linker arm attached to the 5- or other positions of pyrimidines are also suggested in U.S. Pat. No. 4,948,882. Bisulfite-catalyzed transamination of the N⁴-position of cytosine with bifunctional amines is described by Schulman et al. (Nucleic Acids Research, 9(5): 1203-1217, 1981) and Draper et al (Biochemistry, 19: 1774-1781, 1980). By this method, chemical entities are attached via linker arms to cytidine or cytidine-containing polynucleotides. The attachment of biotin to the N4-position of cytidine is disclosed in U.S. Pat. No. 4,828,979, and the linking of moieties to cytidine at the N⁴-position is also set forth in U.S. Pat. Nos. 5,013,831 and 5,241,060. U.S. Pat. No. 5,407,801 describes the preparation of an oligonucleotide triplex wherein a linker arm is conjugated to deoxycytidine via bisulfite-catalyzed transamination. The linker arms include an aminoalkyl or carboxyalkyl linker arm. U.S. Pat. No. 5,405,950 describes cytidine analogs in which a linker arm is attached to the N4-position of the cytosine base.

Numerous cross-linking reagents, including the ones discussed above, are commercially available. Detailed instructions for their use are readily available from the commercial suppliers. A general reference on protein cross-linking and conjugate preparation is: S. S. Wong, Chemistry of Protein Conjugation and Cross-Linking, CRC Press (1991).

Chemical cross-linking may include the use of spacer arms. Spacer arms provide intramolecular flexibility or adjust intramolecular distances between conjugated moieties and thereby may help preserve biological activity. A spacer arm may be in the form of a polypeptide moiety comprising spacer amino acids. Alternatively, a spacer arm may be part of the cross-linking reagent, such as in “long-chain SPDP” (Pierce Chem. Co., Rockford, Ill., cat. No. 21651H).

A variety of coupling or crosslinking agents such as protein A, carbodiimide, dimaleimide, dithio-bis-nitrobenzoic acid (DTNB), N-succinimidyl-S-acetyl-thioacetate (SATA), and N-succinimidyl-3-(2-pyridyldithio) propionate (SPDP), 6-hydrazinonicotimide (HYNIC), N₃S and N₂S₂ can be used in well-known procedures to synthesize targeted constructs. For example, biotin can be conjugated to an oligonucleotide via DTPA using the bicyclic anhydride method of Hnatowich et al. Int. J. Appl. Radiat. Isotop. 33:327 (1982).

In addition, sulfosuccinimidyl 6-(biotinamido)hexanoate (NHS-LC-biotin, which can be purchased from Pierce Chemical Co. Rockford, Ill.), “biocytin”, a lysine conjugate of biotin, can be useful for making biotin compounds due to the availability of a primary amine. In addition, corresponding biotin acid chloride or acid precursors can be coupled with an amino derivative of the therapeutic agent by known methods.

When two of the portions of the targeted construct comprise polypeptides, additional linking techniques are available. For example, recombinant techniques can be used to covalently attach an internalizing polypeptide sequence to a polypeptide targeting moiety or payload, such as by joining the gene coding for the payload with the gene coding for internalizing polypeptide sequence and introducing the resulting gene construct into a cell capable of expressing the conjugate. Alternatively, the two separate nucleotide sequences can be expressed in a cell or can be synthesized chemically and subsequently joined, using known techniques, or the combined sequence may be synthesized chemically as a single amino acid sequence (i.e., one in which both constituents are present) thus obviating any subsequent joining.

Imaging labels may be incorporated into the targeted construct by covalent bonding directly to an atom of the targeting moiety or oligonucleotide, or the label may be non-covalently or covalently associated with the targeting molecule through a chelating structure or through an auxiliary molecule such as mannitol, gluconate, glucoheptonate, tartrate, and the like. When a chelating structure is used to provide spatial proximity between the label and the targeting molecule, the chelating structure may be directly associated with the construct or it may be associated with the construct through an auxiliary molecule such as mannitol, gluconate, glucoheptonate, tartrate, and the like.

Any suitable chelating structure may be used to provide spatial proximity between the radionuclide and the construct through covalent or noncovalent association. Many such chelating structures are known in the art. Preferably, the chelating structure is an N₂S₂ structure, an NS₃ structure, an N₄ structure, an isonitrile-containing structure, a hydrazine containing structure, a HYNIC (hydrazinonicotinic acid)-containing structure, a 2-methylthiolnicotinic acid-containing structure, a carboxylate-containing structure, or the like. In some cases, chelation can be achieved without including a separate chelating structure, because the radionuclide chelates directly to atom(s) in the targeting moiety, for example to oxygen atoms in various moieties.

Radionuclides may be placed in spatial proximity to the targeting molecule using known procedures which effect or optimize chelation, association, or attachment of the specific radionuclide to ligands. For example, when ¹²³I is the radionuclide, the imaging agent may be labeled in accordance with the known radioiodination procedures such as direct radioiodination with chloramine T, radioiodination exchange for a halogen or an organometallic group, and the like. When the radionuclide is ^{99m}Tc, the imaging agent may be labeled using any method suitable for attaching ^{99m}Tc to a ligand molecule. Preferably, when the radionuclide is ^{99m}Tc, an auxiliary molecule such as mannitol, gluconate, glucoheptonate, or tartrate is included in the labeling reaction mixture, with or without a chelating structure. More preferably, ^{99m}Tc is placed in spatial proximity to the targeting molecule by reducing ^{99m}TcO₄ with tin in the presence of mannitol and the targeting molecule. Other reducing agents, including tin tartrate or non-tin reductants such as sodium dithionite, may also be used to make the cardiovascular imaging agent of the invention.

In general, labeling methodologies vary with the choice of radionuclide, the moiety to be labeled and the clinical condition under investigation. Labeling methods using ^{99m}Tc and ¹¹¹In are described for example in Peters, A. M. et al., Lancet 2: 946-949 (1986); Srivastava, S. C. et al., Semin. Nucl. Med. 14(2):68-82 (1984); Sinn, H. et al., Nucl. Med. (Stuttgart) 13:180, 1984); McAfee, J. G. et al., J. Nucl. Med. 17:480-487, 1976; McAfee, J. G. et al., J. Nucl. Med. 17:480-487, 1976; Welch, M. J. et al., J. Nucl. Med. 18:558-562, 1977; McAfee, J. G., et al., Semin. Nucl. Med. 14(2):83, 1984; Thakur, M. L., et al., Semin. Nucl. Med. 14(2):107, 1984; Danpure, H. J. et al., Br. J. Radiol., 54:597-601, 1981; Danpure, H. J. et al., Br. J. Radiol. 55:247-249, 1982; Peters, A. M. et al., J. Nucl. Med. 24:39-44, 1982; Gunter, K. P. et al., Radiology 149:563-566, 1983; and Thakur, M. L. et al., J. Nucl. Med. 26:518-523, 1985. An example of labelling with ¹²⁵I is described in detail in the Exemplification below.

Synthesized targeted constructs can be characterized using standard methods of high field NMR spectra as well as IR, MS, and optical rotation. Elemental analysis, TLC, and/or HPLC can be used as a measure of purity. A purity of at least about 80%, preferably at least about 90%; more preferably at least about 95% and even more preferably at least about 98% is preferred. TLC and/or HPLC can also be used to characterize such compounds.

Once prepared, candidate targeted constructs can be screened for ability to bind the corresponding target, for in vivo binding to sites of infection, or in vitro or in vivo binding to tumors. The internalization and retention of a target construct in a cell can be determined, e.g., as described in the Examples. In addition, stability of a targeting construct can be tested by incubating the compound in serum, e.g., human serum, and measuring the potential degradation of the compound over time. Stability can also be determined by administering the compound to a subject (human or non-human), obtaining blood samples at various time periods (e.g., 30 min, 1 hour, 24 hours) and analyzing the blood samples for derived or related metabolites.

V. Administration of Targeted Constructs

For use in therapy, an effective amount of an appropriate targeted construct can be administered to a subject by any mode which allows the compound to be taken up by the appropriate target. Preferred routes of administration include oral and transdermal (e.g., via a patch). Examples of other routes of administration include injection (subcutaneous, intravenous, parenteral, intraperitoneal, intrathecal, etc.). The injection can be in a bolus or a continuous infusion.

Pharmaceutical compositions of the invention include a pharmaceutical carrier that may contain a variety of components that provide a variety of functions, including regulation of drug concentration, regulation of solubility, chemical stabilization, regulation of viscosity, absorption enhancement, regulation of pH, and the like. The pharmaceutical carrier may comprise a suitable liquid vehicle or excipient and an optional auxiliary additive or additives. The liquid vehicles and excipients are conventional and commercially available. Illustrative thereof are distilled water, physiological saline, aqueous solutions of dextrose, and the like. For water soluble formulations, the pharmaceutical composition preferably includes a buffer such as a phosphate buffer, or other organic acid salt, preferably at a pH of between about 7 and 8. For formulations containing weakly soluble compounds, micro-emulsions may be employed, for example by using a nonionic surfactant such as polysorbate 80 in an amount of 0.04-0.05% (w/v), to increase solubility. Other components may include antioxidants, such as ascorbic acid, hydrophilic polymers, such as, monosaccharides, disaccharides, and other carbohydrates including cellulose or its derivatives, dextrins, chelating agents, such as EDTA, and like components well known to those in the pharmaceutical sciences, e.g., Remington's Pharmaceutical Science, latest edition (Mack Publishing Company, Easton, Pa.).

Targeted constructs of the invention include pharmaceutically acceptable salts thereof, including those of alkaline earths, e.g., sodium or magnesium, ammonium or tetraalkylammonium. Other pharmaceutically acceptable salts include organic carboxylic acids such as acetic, lactic, tartaric, malic, isethionic, lactobionic, and succinic acids; organic sulfonic acids such as methanesulfonic, ethanesulfonic, and benzenesulfonic; and inorganic acids such as hydrochloric, sulfuric, phosphoric, and sulfamic acids. Pharmaceutically acceptable salts of a compound having a hydroxyl group include the anion of such compound in with a suitable cation such as sodium, ammonium, or the like.

The targeted constructs are preferably administered parenterally, most preferably intravenously. A preferred formulation for intravenous injection should contain, in addition to the targeted construct, an isotonic vehicle such as Sodium Chloride Injection, Ringer's Injection, Dextrose Injection, Dextrose and Sodium Chloride Injection, Lactated Ringer's Injection, or other vehicle as known in the art. Alternatively, the construct may be administered subcutaneously via controlled release dosage forms.

In addition to administration with conventional carriers, the targeted constructs may be administered by a variety of specialized oligonucleotide delivery techniques. Sustained release systems suitable for use with the pharmaceutical compositions of the invention include semi-permeable polymer matrices in the form of films, microcapsules, or the like, comprising polylactides; copolymers of L-glutamic acid and gamma-ethyl-L-glutamate, poly(2-hydroxyethyl methacrylate), and like materials, e.g., Rosenberg et al., International application PCT/US92/05305.

The targeted constructs may be encapsulated in liposomes for therapeutic delivery, as described for example in Liposome Technology, Vol. 11, Incorporation of Drugs, Proteins, and Genetic Material, CRC Press. The targeted constructs, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, ionic surfactants such as diacetylphosphate, stearylamine, or phosphatidic acid, and/or other materials of a hydrophobic nature.

A preferred dose for treating or preventing a tumor or site of infection is in the range of 5 μg-100 mg. However, the exact dose depends to a great extent on the toxicity of the therapeutic agent being administered. For example, a subject cannot withstand more than a milligram dose of bleomycin. In addition, certain chemotherapeutic peptides cause hemophilia and other blood disorders when given to a subject in microgram amounts. However, the selective targeting of a therapeutic agent by the instant targeted constructs decreases their otherwise toxic effects on normal body cells.

VI. Use of Imaging Targeted Constructs

Targeted constructs that have been labeled with an appropriate imaging agent can be added to a particular tumor cell line, tissue type, or bacteria-, virus-, or fungus-infected tissue culture to test the binding affinity of a particular candidate targeted therapeutic. Labeled targeted constructs can also be injected into an appropriate subject (e.g., monkey, dog, pig, cow) and its binding with tumors, tissue types, or sites of infection in vivo can then be monitored.

Imaging agents of the invention may be used in accordance with the methods of the invention by one of skill in the art, e.g., by specialists in nuclear medicine, to image sites of infection or inflammation in a subject. Any site of infection or inflammation may be imaged using the imaging agents of the invention.

Images can be generated by virtue of differences in the spatial distribution of the imaging agents which accumulate at a site of tumor, infection, or inflammation. The spatial distribution may be measured using any means suitable for the particular label, for example, a gamma camera, a PET apparatus, a SPECT apparatus, and the like. Some lesions may be evident when a less intense spot appears within the image, indicating the presence of tissue in which a lower concentration of imaging agent accumulates relative to the concentration of imaging agent which accumulates in surrounding tissue. Alternatively, a lesion may be detectable as a more intense spot within the image, indicating a region of enhanced concentration of the imaging agent at the site of the lesion relative to the concentration of agent which accumulates in surrounding tissue. Accumulation of lower or higher amounts of the imaging agent at a lesion may readily be detected visually. Alternatively, the extent of accumulation of the imaging agent may be quantified using known methods for quantifying radioactive, fluorescent, or other emissions. A particularly useful imaging approach employs more than one imaging agent to perform simultaneous studies.

Preferably, a detectably effective amount of the imaging agent of the invention is administered to a subject. In accordance with the invention, “a detectably effective amount” of the imaging agent of the invention is defined as an amount sufficient to yield an acceptable image using equipment which is available for clinical use. A detectably effective amount of the imaging agent of the invention may be administered in more than one injection. The detectably effective amount of the imaging agent of the invention can vary according to factors such as the degree of susceptibility of the individual, the age, sex, and weight of the individual, idiosyncratic responses of the individual, the dosimetry. Detectably effective amounts of the imaging agent of the invention can also vary according to instrument and film-related factors. Optimization of such factors is well within the level of skill in the art.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

Diseases and conditions that can be treated according to the invention include any conditions in which it is desirable to kill certain cells or to slow down or inhibit their proliferation. In such embodiments, the payload of the targeting construct can be a toxin, which kills cells. Such conditions include those resulting from excessive or uncontrolled cell growth, such as in benign and malignant cancer. Generally, any type of proliferative disease or condition can be treated (i.e., to improve at least one symptom of the disease or condition) with the targeting constructs of the invention. Other diseases that can be treated include auto-immune diseases and viral infections.

Diseases can be treated by administration of a targeted oligonucleotide of the invention to a subject. Alternatively, targeted oligonucleotides can be administered ex vivo into cells, e.g., cells of a subject. Accordingly, in one embodiment, the invention provides a method for treating a subject having a disease, comprising obtaining cells from the subject, contacting the cells ex vivo with a targeting construct of the invention, and introducing the cells back into the subject. Ex vivo administration of the targeted construct of the invention can also be used for imaging purposes, rather than for treatment purposes.

The invention can be used to treat numerous types of cancers, including solid tumors as well as cancers of blood cells, lymphomas and leukemias. Solid tumor cancers include ovarian, breast, colorectal, melanoma, pancreas, stomach, gall baldder, oesophagus, lung, gliomas, renal, and thyroid cancers. Genes specifically expressed in these tumors are set forth, e.g., in U.S. Pat. No. 6,093,399. For example, target genes against which targeting constructs can be directed for treating breast cancer include bcl-1, bcl-2, vasopressin related proteins; see, North, et al., Breast Cancer Res. Treat., 34(3):229-35 (1995); Hellemans, Br. J. Cancer, 72(2):354-60 (1995); and Hurlimann, et al., Virchows Arch., 426(2). 163-8 (1995)). Genes for targeting other carcinomas include, e.g., c-myc, int-2, hst-1, ras and p53 mutants; see, Issing, et al., Anticancer Res., 13(6B):2541-51 (1993); Tjoa, et al., Prostate, 28(1):65-9 (1996); Suzich, et al., Proc. Natl. Acad. Sci. USA, 92(25):11553-7 (1995); and Gjertsen, et al., Lancet, 346(8987):1399-400 (1995)). Genes for targeting in B cell lymphomas include CD19, CD20, CD37, as well as a gene described in U.S. Pat. No. 6,099,846. A gene associated with renal carcinoma is RAGE (Gaugler et al. (1996) Immunogenetics 44:323). Genes associated with prostate cancer include prostate specific membrane antigen (PSMA) (U.S. Pat. No. 5,538,866); prostate specific antigen (PSA) (Watt K W et al., Proc Natl Acad Sci USA (1986) 83:3166-3170); and prostatic acid phosphatase (PAP) (Sharief, F. S., et al., Biochem Biophys Res Commun (1989) 180:79-86; Tailor, P. G., et al., Nucleic Acids Res (1990) 18:4928). For treating cancer of the pancreas or colorectal cancer, the target gene can be carcinoembryonic antigen (CEA) (see, e.g., Benchimol, et al., Cell, 57:327-324, 1989). For treating melanomas, the targeted gene can be melanocyte differentiation antigen -MART-1/Melan A (Coulie et al., 1994, J. Exp. Med. 180:35; Hawakami et al., 1994, PNAS 91:3515; Bakker et al., 1994, J. Exp. Med. 179: 1005), gplO0, tyrosinase/albino, p97 melanoma antigen, and any of the various MAGEs (melanoma associated antigen E), including MAGE 1, 2, 3, 4, etc. (Boon, T. Scientific American (March 1993):82-89; e.g., Zhai, et al., J. Immunol., 156(2):700-10 (1996); Kawakami, et al., J. Exp. Med., 180(1):347-52 (1994); and Topalian, et al., Proc. Natl. Acad. Sci. USA, 91(20):9461-5 (1994)). Since at least some of the above-described genes encode membrane proteins, the targeting moiety of the targeting construct can also be directed to these membrane proteins. Thus, in certain embodiments, the targeting moiety and the nucleic acid will be targeted to the same gene or protein.

For treating viral infections, targeting constructs can include a nucleic acid encoding a viral protein. Viruses that result in chronic infections include the hepadnaviruses (including HBV), the lentiviruses (including HIV), herpesviruses (including HSV-1, HSV-2, EBV, CMV, VZV, and HHV-6), and the flaviviruses/pestiviruses (including HCV), and human retroviruses, for example, human T lymphotropic viruses (HTLV-1 and HTLV-2) that cause T cell leukemia and myelopathies. Other organisms that cause chronic infections which can also be treated according to the invention include, for example, pathogenic protozoa, (e.g., Pneumocystis carinii, trypanosoma, malaria and Toxoplasma gondii), bacteria (e.g., mycobacteria, salmonella and wisteria) and fungi (e.g. candida and aspergillus). The nucleotide sequences of a number of these viruses, including different species, strains, and isolates are known in the art. For reviews see: Robinson (1990) (Hepadnaviridae); Levy, Microbiological Reviews, 57:183-289 (1993) (HIV); and Choo et al., Seminars in Liver Disease, 12:279-288 (1992) (HCV).

For example, for treating infections by herpesvirus family viruses, including herpes simplex virus (HSV) types 1 and 2, such as HSV-1 and HSV-2, the target construct can include an oligonucleotide complementary to a gene encoding glycoprotein gB, gD or gH; genes from varicella zoster virus (VZV), Epstein-Barr virus (EBV) and cytomegalovirus (CMV) include CMV gB and gH; and genes from other human herpesviruses include HHV6 and HHV7. (See, e.g. Chee et al., Cytomegaloviruses (J. K. McDougall, ed., Springer-Verlag 1990) pp. 125-169, for a review of cytomegalovirus genes; McGeoch et al., J. Gen. Virol. (1988) 69:1531-1574, for a discussion of the various HSV-1 genes; U.S. Pat. No. 5,171,568 for a discussion of HSV-1 and HSV-2 gB and gD genes; Baer et al., Nature (1984) 310:207-211, for the identification of genes in an EBV genome; and Davison and Scott, J. Gen. Virol. (1986) 67:1759-1816, for a review of VZV.)

Infections by the hepatitis family of viruses, such as hepatitis A virus (HAV), hepatitis B virus (HBV), hepatitis C virus (HCV), the delta hepatitis virus (HDV), hepatitis E virus (HEV) and hepatitis G virus (HGV) can also be treated as described herein. By way of example, the viral genomic sequence of HCV is known, as are methods for obtaining the sequence. See, e.g., International Publication Nos. WO 89/04669; WO 90/11089; and WO 90/14436. The HCV genome encodes several viral proteins, including E1 (also known as E) and E2 (also known as E2/NSI) and an N-terminal nucleocapsid protein (termed “core”) (see, Houghton et al., Hepatology (1991) 14:381-388, for a discussion of HCV proteins, including E1 and E2). Genes encoding each of these proteins can be targeted with the targeting constructs described herein for treating viral infections.

It is expected that, even if the targeting construct enters cells that are not infected by a virus, the targeting construct will be retained more efficiently in virus-infected cells.

The invention also provides methods for selectively modifying (e.g., killing or labeling) specific cells in a cell population. In one embodiment, the cell population is in vitro. The cell population can have been obtained from a subject. The cell population can be incubated with a targeting construct of the invention in which the targeting moiety binds specifically with a cell membrane protein, e.g., a receptor, of the target cells (i.e., those cells in the population that one desires to modify) and in which the nucleic acid moiety is complementary to a gene that is expressed, preferably at high levels, in the target cell type. Following incorporation of the targeting construct in at least some target cells, the cell population, or a fraction thereof, can be administered to a subject, who can be the same or different from the one from whom the cell population had been obtained.

In one embodiment, specific lymphocytes are eliminated from blood cells or from bone marrow cells of a subject. Accordingly, blood cells or bone marrow cells of the subject are obtained, incubated in vitro in the presence of a targeting construct, in which the targeting moiety specifically binds to the lymphocytes to eliminate, but essentially not to other cells in the sample (or at least to other important cells in the sample), and the targeting construct further comprises a therapeutic compound for killing the target cells or for inhibiting their proliferation. The targeting moiety can be a ligand that binds to specific T lymphocyte receptors, e.g., those binding to a self-antigen, in which case, the ligand can be the self-antigen, or a portion thereof having an epitope of the self-antigen recognized by the T cell receptor. The therapeutic compound could be a toxin, such as aflatoxin. Incubation of the cells with the targeting construct can be conducted for a time sufficient to permit a significant amount of the target lymphocytes to have incorporated the target construct. The time of incubation can be determined by monitoring the amount of target lymphocytes remaining in the population during the time of incubation. Following incubation, the population of cells can be administered back to the subject. It will be understood that the targeting constructs can also be administered to a subject.

Accordingly, the invention provides methods for treating auto-immune diseases, e.g., insulin-dependent diabetes mellitus (IDDM) (auto-antigens are islet cell antigens, including glutamic acid decarboxylase); myasthenia gravis (auto-antigen is the acetylcholine receptor); and autoimmune thyroiditis or Graves disease (thyroid follicular epithelial cell auto-antigens). Other potential auto-immune diseases include multiple sclerosis; lupus erythematosous, rheumatoid arthritis, ALS (Lou Gehrig's disease), and interstitial cystitis and prostatitis.

The targeting compounds of the invention can also generally be used in any inflammatory disease, in which one desires to inhibit growth of, or destroy, lymphocytes that are responsible for or aggravate the inflammatory disease.

The present invention is further illustrated by the following examples which should not be construed as limiting in any way. The contents of all cited references including literature references, issued patents, published or non published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); , Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

Exemplification

### Example 1

### Preparation of Derivatized Oligonucleotides

This Example describes a convenient technique for radiolabelling nucleic acid molecules at the 5′ end.

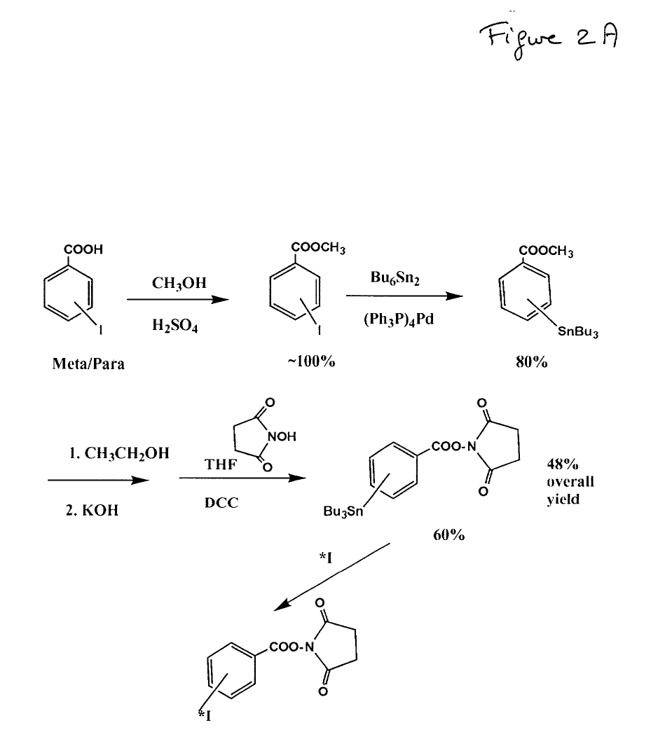

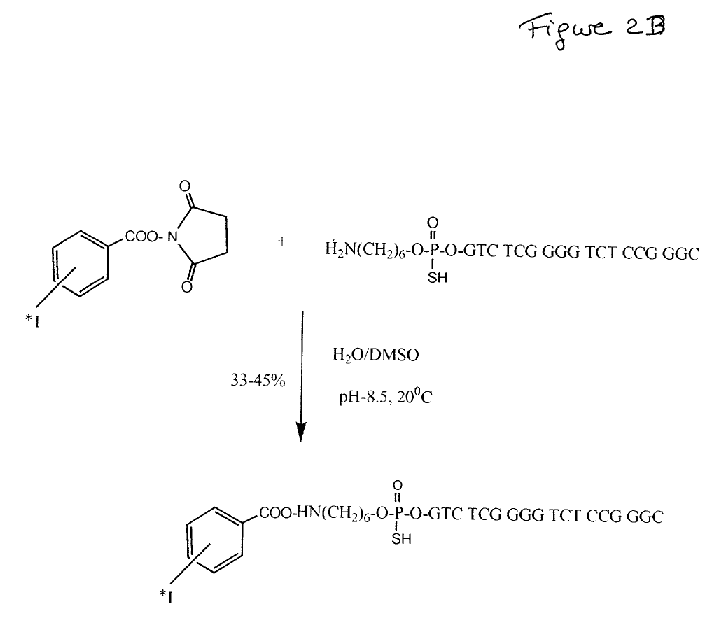

c-myb-octadecamer oligonucleotides (antisense Y-GTG-TCG-GGG-TCT-CCG-GGC (SEQ ID No. 6)) and sense Y-GCC-CGG-AGA-CCC-CGA-CAC (SEQ ID No. 7)) were synthesized and derivatized at the 5′ end with hexylaminophosphothioate (Y═NH₂-(CH₂)₆—O—P(═O)(SH)O—) in excellent yield and purity, as described in details below. These derivatized c-myb oligomers were then radioiodinated by reaction with p-iodo[¹²⁵I]-N-succinimidyl benzoate in DMSO/water which was prepared in excellent yield and purity from the corresponding p—tributyltin-N-succinimidylbenzoate (FIGS. 2A and B), also as described in details below. Stable iodide was added to achieve appropriate specific activity for optimizing cellular uptake. This in turn increased the labeling efficiency of the ODN's.

Oligonucleotide Synthesis

Oligonucleotide phosphorothioates were prepared with an automated synthesizer (Biosearch 8700, Milligen, Bedford, IVIA) by standard phosphoroamidite chemistry. N-Monomethoxytrityl aminohexa-6-oxy-cyanoethyl-N,N-diisopropylamino phosphoroamidite (Millipore) was used for the final coupling in order to derivatize the 5′-end according to the manufacturers protocol. Phosphorothioate bonds were introduced by oxidization with the Beaucage thiolating reagent (Iyer, R. P., Uznanski, B., Boal, J., Storm, C., Egan, W., Matsukura, M., Broder, S., Zon, G., Wilk, A. & Koziolkiewicz M. (1990) Nucleic Acids Res. 18, 2855-2859). The crude oligonucleotides, “MMT-on” phosphorothioates, were purified by chromatography on a C-18 reversed phase column (1×20 cm) employing a gradient of acetonitrile (Buffer A: 0.1 M ammonium acetate, Buffer B: 80% acetonitrile: 20% buffer A, v/v). The elution conditions were: 100% A for 2 min. followed by a linear gradient to 80% B over 45 min at a constant flow rate of 2 ml/min. After removal of the monomethoxytrityl group with 80% acetic acid, the oligonucleotides were dialyzed against distilled water (36-40 h, Spectra-Por membrane, molecular weight cut-off: 3500 Da). HPLC on a WAX-column and PAGE electrophoresis (20% gel) showed a single species. (Metelev, V. & Agrawal, S. (1992) Anal Biochem 200,342-346).

Preparation of p-Tri-N-butylstanylbenzoic acid N-hydroxysuccinimide ester (p-BuATE)

A solution of para-iodobenzoic acid (20.0 g), methanol (200 mL) and concentrated sulfuric acid (5 mL) was heated and refluxed for 20 h, cooled, concentrated to 50 mL and poured over 400 mL of 1 N sodium bicarbonate. The mixture was extracted with ether (3×300 mL) and the ether layer was washed with water, dried over anhydrous magnesium sulfate and evaporated to dryness to yield methyl para-iodobenzoate as an off-white solid that was a single component by TLC on silica gel (hexane:ethyl acetate 95:5, Rf: 0.7).This product was used without further purification.

Methyl para-iodobenzoate (5.25 g, 20 mmol), hexabutylditin (17.5 g, 30 mmol) and tetrakis(triphenylphosphine) palladium oxide (0.22 g, 0.2 mmol) in dry toluene (50 mL) were heated under nitrogen at 110 C for 24 h. The reaction solution was cooled, decanted and evaporated to dryness. The thick, oily residue was purified by column chromatography on silica gel (230 g) using hexane:ethyl acetate (9:1) as the eluent. The fractions containing the pure product were combined and evaporated to dryness to give 6.85 g (16 mmol, 80% yield) of a clear oil.

Potassium hydroxide (1.15 g, 20 mmol) was added to a solution of methyl p-tributylstannylbenzoate (6.7 g, 16 mmol) in ethanol (150 mL) and the mixture was heated for 6 h, cooled to room temperature and poured into ice water (50 mL) containing 1.6 g of acetic acid. The mixture was extracted with ether (3×250 mL) and the ether layer was washed with brine, dried over anhydrous magnesium sulfate and evaporated to dryness. The resultant oil (6.2 g) was used in the next step without purification.

The oil (6.2 g) was dissolved in 50 mL of dry tetrahydrofuran (THF) and dicyclohexyl carbodiimide (3.64 g) and N-hydroxysuccinimide (2.04 g) were added sequentially. The reaction mixture was maintained at 40° C. for 20 h, filtered to remove dicyclohexylurea and evaporated to dryness. The oily residue was purified by column chromatography on silica gel (190 g) using hexane:ethyl acetate (3:1) as the eluant. The product was concentrated to give a clear oil (4.9 g, 9.5 mmol) in a 60% yield for the two steps and an overall yield of 48%. The procedure for preparing this compound is summarized in FIG. 2A. Meta tri-n-butylstannylbenzoic acid and N-hydroxysuccinimide ester were prepared essentially in the same manner. For all products, NMR spectra were identical to those previously reported (Hanson, R. N., Franke, L., Lee, S. H. & Seitz, D. E. (1987) Int. J. Rad. Appl. Instrum. [A] 38, 641-645).

Preparation of N-Succinimidyl-p-[¹²⁵I]-Benzoate (p-IBTE)

Radioiodination of p-BuATE was performed as described previously (Zalutsky, M. R. & Narula, A. S. (1988) Int J. Rad. Appl. Instrum. [A] 39, 227-232) with some modification. Typically, 10-20 μl of Na¹²⁵I (400-500 μl Ci, Amersham, 2,5003,000 μCi/mole in NaOH, pH: 7-8.5), 10 μl of Kl (0.04 N) in water, 5-15 μl of 2% acetic acid in chloroform, 10 μl of t-butyl hydroperoxide in chloroform (1 M) and 200 μg of p-BuATE in chloroform (2 mg/ml), were placed in a 5 ml reactive vial. The reaction was allowed to proceed at room temperature with stirring for 20 min. and quenched with 10 μl of 10% KF, 10 μl of saturated NaHSO₃ and 10 μl of saturated Na₂CO₃. The mixture was extracted with CH₂CI₂ (3×1 ml) and dehydrated over a column (4×0.4 cm, pasture pipet) containing Na₂SO₄. The solvent was evaporated to dryness under a stream of nitrogen and the residue was dissolved in 300 μl of chloroform and loaded onto a 0.5 g silica gel column (Supelco, Inc.) that was prewashed with 10 ml of chloroform. The fractions (3×0.5 ml, 2-5) containing the radiolabeled compound were combined and evaporated to dryness with a stream of nitrogen, Radiochemical purity as determined by TLC silica gel (hexane:ethyl acetate 3:2, R_{f=}0.4) using a radioactivity strip scanner (Bioscan) was >96%. Radiochemical yield ranged between 40-56%.

Preparation of [¹²⁵I] c-myb Oligonucleotides

The 5′ amino-derivatized oligonucleotides (“S-ODNs”) (0.4-0.5 mg) were dissolved in 50 μl of a sterile solution of 0.1 N NaHCO₃, pH 8.5. Radioiodinated p-IBTE dissolved in 50 μl of DMSO was added and the mixture was allowed to react for one hour at 150° C. The radiolabeled S-ODN's were purified by two cycles of size exclusion chromatography on Sephadex G-25 (Pharmacia, Inc.) eluted with PBS. Aliquots (0.7 ml) were monitored for UV absorbance (OD₂₆₀ nm) and radioactivity before combining the desired fraction. The procedure for preparing this compound is illustrated in FIG. 2B. Stock solutions of S-ODN's (sense and antisense c-myb) were adjusted to 96 μM by the addition of a quantity of unlabeled material which was determined by absorbance at 260 nm, taking into account the molar extinction coefficient of the nucleotides present in each sequence (31). 10 μl aliquots of each solution contained approximately 22,000 CPM (15.4 mCi/mmol). For the biodistribution experiment in rats, radiolabeled C-myb antisense was not diluted with unlabeled compound; specific activity (7,400 mCi ¹²⁵I/mmole of ODN).

Derivatization of the 6-position of the oligonucleotide phosphorothioates with a hexylamino tether represents a general and convenient method for specific radiolabeling of the oligomers. The free amino group facilitates rapid nucleophilic attack of the activated ester in hydrophobic solvents such as DMSO. At room temperature, the reaction was completed within 1 h and the radiolabeling yield was relatively high (>40%) (FIG. 2B). The yield decreased when aqueous solutions were used to dissolve the activated ester (<6%). Under these conditions, lower temperature and longer incubation periods did not improve the yield. The simple purification with an ion exchange column resulted in high radiochemical purity (>96%).

### Example 2

### Radiostablilty of 5′-p-iodo-[¹²⁵I]-benzoate-derivatized c-mvb

The radiolabeled compounds were stored at −20° C. for 6 months. During this time, the solutions were thawed 9 times and kept at temperatures between 4-10° C. for 1-2 hours. The integrity of the product was then evaluated by C-18 RP HPLC (Microsorb; 5 m , 25 cm×4.6 mm). The mobile phase consisted of: Buffer A: 0.1 N Na acetate pH 6.3, Buffer B-Acetonitrile. The elution conditions were: 100% A, 0-3 min.; 0-50% B (3-30 min.), flow rate=1.5 ml/min. Absorbance was monitored at 254 nm. ¹²⁵I-radioactivity was measured by counting each fraction (0.75 ml) in a well counter (LKB). As shown in FIG. 3, the radiolabeled compound was stable for up to at least 6 months when stored at −20° C.

### Example 3

### I-c-myb Phosphorothioate Stability in Human Serum

Human serum was diluted with 0.9% NaCl to 60% (WV) and filtered with a 0.2 gm Teflon filter. Twenty-five μl (96 M, 2,200 CPM/1) of S-ODN (sense c-myb) was added to 80 μl of the serum solution to yield a final concentration of 22 μM. Aliquots of this mixture were incubated for 0, 1, 2 and 4 hours in a water bath at 36-37° C. and samples were stored frozen at −20° C. until analysis.

The column and eluants used for analysis were the same as described above. The elution profile was modified to flush proteins from the column prior to the elution of ODN. The elution conditions were: 100% A (0-10 min.), 0-100% B (11-30 min.), 100% B (30-35 min.), flow rate=1.5 m/min. Radioactivity was measured in aliquots, each collected for 0.5 min.

The results indicated that incubation of ¹²⁵I-c-myb phosphorothioate (2 μM) with human serum (45%) at 36-37° C. for up to 4 hrs. resulted in minimal deiodination (<2%); peak eluting at 1-2 min. HPLC analysis of the fractions showed that most of the radioactivity was associated with the UV-active oligonucleotide molecule. As expected, HPLC analysis suggested that the major site for degradation of the radiolabeled oligonucleotide was at the 3′-end (peak eluting between 17-18 min). The degradation product in human serum increased by 6.4% after 4 hours of incubation.

Targeting active antisense oligonucleotides against malignant tissue for either imaging or therapeutic applications could be improved by positioning the radiolabel at the 5′-end, and stability is improved by blocking the 3′-end as previously described (Ausubel, F. M., Brent, R., Kingston, R. E., Moore, D. D., Seidman, J. G., Smith, J. A., Struhl, K., Albright, L. M., Coen, D. M., & Varki, A. (1987) In: Current protocols in molecular biology. (J. Wiley, New York), pp. A.3D. 1-8). The stability of the radioiodinated octadecamer in serum is in good agreement with the results of studies with tritiated S-ODN's (Temsamani, J. et al., Antisense Res. Dev. 1993, 3, 277-284; Agrawal, S. et al., Proc. Natl. Acad. Sci. USA 1991, 88, 7595-7599). The minor degree of deiodination that was detected in vitro and in vivo indicates that the radiolabeling method yields metabolically stable radiopharmaceuticals. The in vivo stability of the radiolabel should simplify the interpretation of imaging studies.

### Example 4

### In vitro Uptake of Oligonucleotides as a Function of Time

This Example describes the uptake of the labeled c-myb oligonucleotides as a function of time in three different cell lines.

NIH-3T3 mouse fibroblasts, human neuroblastoma (SK-N-SH) and human intestinal smooth muscle cells (HISM) were obtained from the American Type Culture Collection (ATCC), 10801 University Blvd., Manassas, Va. 20110. Cells were expanded in 75 cm² flasks under 5% CO₂/95% air in Eagle's MEM or DMEM containing 10% (V/V) fetal calf serum and penicillin/streptomycin. Other supplements were added according to the instructions of the ATCC. Cells were seeded in 12 well plates, 36-48 hours prior to each experiment to give a final cell number of about 10⁵ cells/well for the NIH-3T3 and SK-N-SH cell lines and about 2.5-3×10⁴ cells/well for the slower growing HISM cells. Subconfluent monolayers were used.

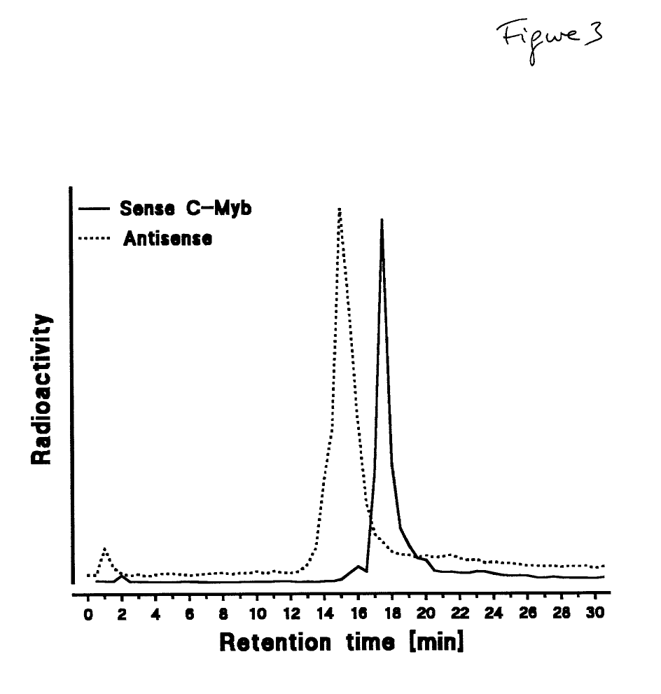

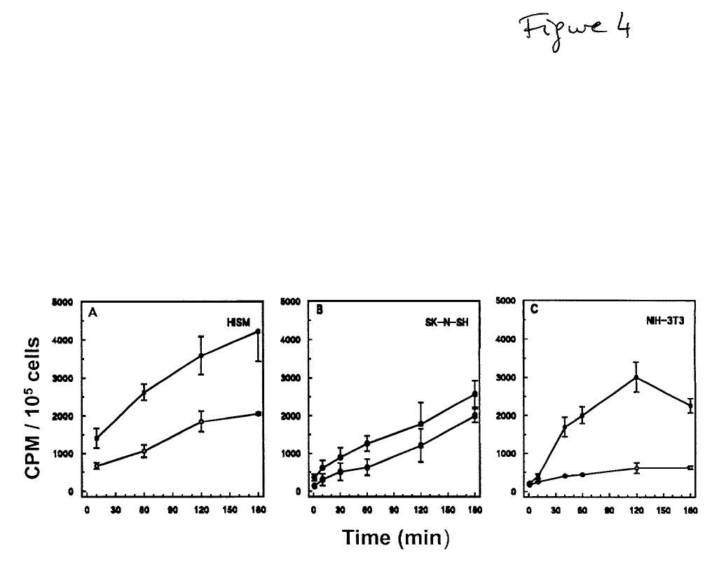

On the day of each experiment, the incubation medium was replaced with fresh DMEM containing 10% FCS (to remove detached or dead cells) and the plates were incubated at 37° C. for 2 hours. The medium was then replaced with 350 μl of DMEM containing 10 mM HEPES buffer and radiolabelled c-myb phosphorothioate analog at a final concentration of 5 μM. To assure constant conditions, the media were preincubated at 37° C. in a 5% CO₂ containing atmosphere. All studies were performed at least twice in triplicate wells. The cells were incubated with the oligonucleotides for the times indicated in FIG. 4.

To determine the concentration of S-ODN's radioactivity that was associated with cells at the end of each incubation period (uptake kinetics), the wells were washed with 1.0 ml of ice cold phosphate buffered saline (PBS, 3×) to remove extracellular radioactivity, followed by lysis with 0.5 ml of 1 N NaOH. The cell lysates were pooled with subsequent water washes (0.5 ml) after incubation at 37° C. for at least 2 hours. ¹²⁵I radioactivity was measured with a well-type automatic gamma counter. Cell number was determined from parallel wells that were washed with warm PBS (×3) followed by trypsinization.

Statistical analysis was performed by one- or two-way Analysis of Variance (ANOVA). Individual means were compared by Student's t-test with correction for multiple comparisons. All results are expressed as meanisem. P values of <0.05 were considered to be significant.

The uptake kinetics for sense and antisense ¹²⁵I-c-myb phosphorothioates by HISM, SK-N-SH and NIH-3T3 cell lines demonstrated in all cases increased cellular uptake as a function of incubation time (FIG. 4). In all three cell lines, uptake of the sense form of c-myb phosphorothioate was lower than that of the corresponding antisense compound. As expected, the largest difference between sense and antisense uptake was observed with HISM cells (FIG. 4A). This cell line is known to express c-myb. Since nonspecific uptake of oligonucleotides has been observed in many cell lines (Agrawal, S. Antisense Therapeutics, in Current Opinion in Chemical Biology, Vol. 2, 1998, pp. 519-528), nonspecific uptake in the neuroblastoma cells and fibroblasts is not surprising.

### Example 5

### In vitro Uptake of Oligonucleotides as a Function of Concentration

This Example describes the uptake of the labeled c-myb oligonucleotides as a function of oligonucleotide concentration in three different cell lines.

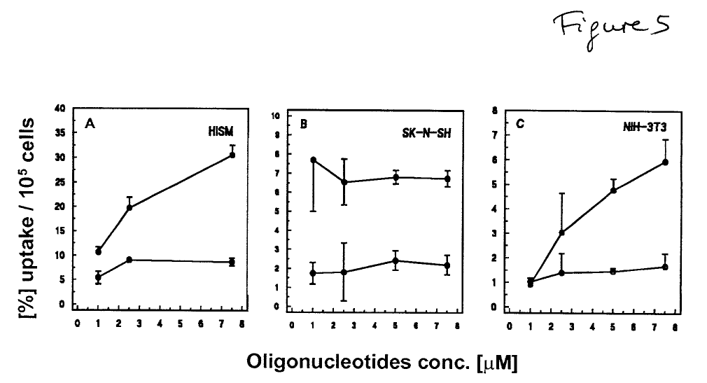

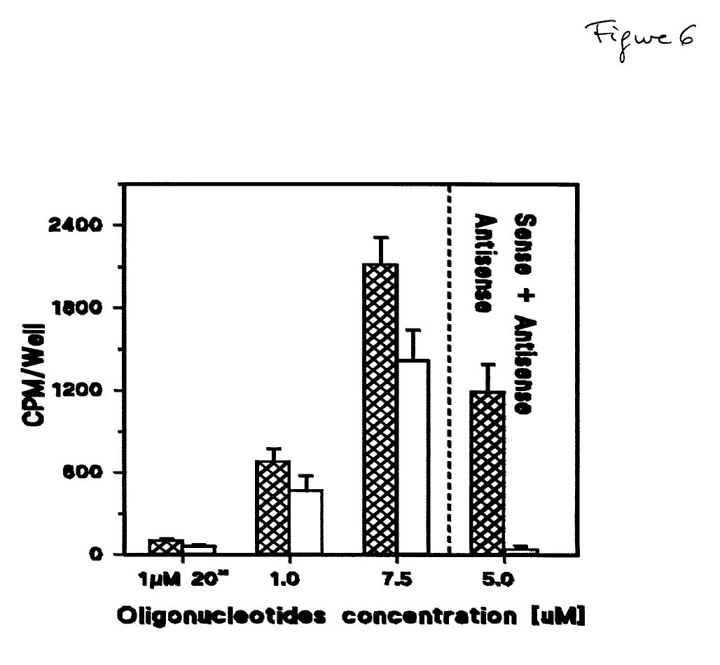

HISM, SK-N-SH and NIH-3T3 cells were incubated with radiolabed c-myb sense and antisense oligonucleotides as described above, except that all incubations with the oligonucleotides were done for 40 minutes and the concentrations were as indicated in FIG. 5.

The results indicate that HISM cells showed a marked increase in uptake with increasing concentration of radiolabeled antisense (about 10% at 1 μM and about 30% at 7.5 μM), whereas the labeled sense showed only a slight change (about 5% at 1 μM versus about 7% at 7.5 μM) (FIG. 5A). With the neuroblastoma cell line there was no change in antisense uptake over the concentration range studied (about 7% for 1-7.5 μM concentrations) (FIG. 5B). Over the same concentration range, the sense compound showed lower uptake (about 2%). For the fibroblast cell line, the percent uptake at 1 μM was similar for both sense and antisense (about 4%) (FIG. 5C). This uptake decreased with increasing sense concentration (−1% at 7.5 μM) while with the antisense, uptake increased to about 6% at 5 μM and than decreased slightly to about 5.5% at 7.5 μM. These findings are consistent with the results of studies performed with tritiated oligonucleotides.

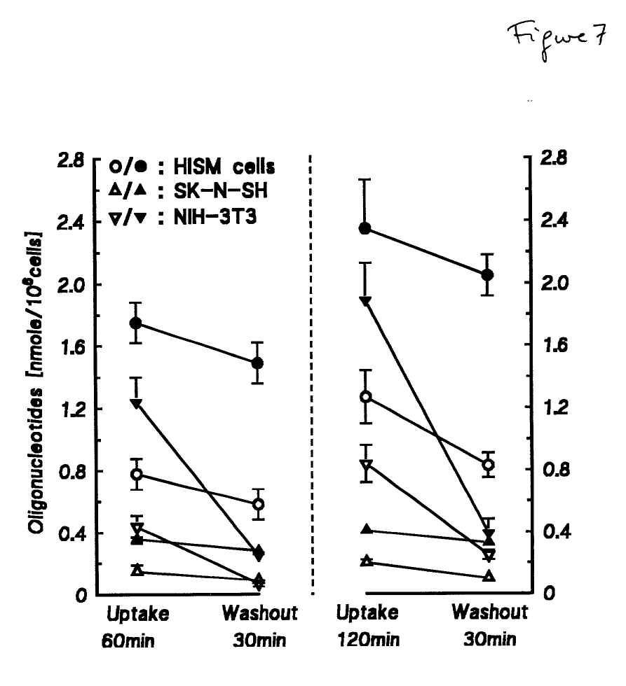

FIG. 6 also shows the amount of radiolabeled sense and antisense c-myb oligonucleotides that were incorporated into SK-SN-NH cell line, after 20 seconds incubation with 1 μM oligonucleotide concentration (first two columns) or after 40 minutes incubation at 1 or 7.5 μM oligonucleotide concentration (columns 3-6 of FIG. 6).

Co-incubating aliquots of sense and antisense stock solutions at equimolar concentrations for 10 minutes at room temperature prior to incubation with SK-S-NH cells at 5 μM final total concentration for 40 minutes, reduced cellular bound radioactivity to nearly background levels (last column of FIG. 6). As a comparison, the same cells were incubated with 5 μM antisense c-myb for 40 minutes (second to last column of FIG. 6). These results indicates a degree of specificity of the transporter system (Loke, S. L. et al., Proc. Natl. Acad. Sci. USA, 1989, 86, 3474-3478; Stein, C. A. et al., Biochemistry, 1993, 32, 4855-4861) for single stranded S-ODN's and an important role of molecular charge on cellular binding and uptake.

### Example 6

### Uptake and Retention of Oligonucleotides

This Example demonstrates that retention of antisense oligonucleotides is higher than retention of sense oligonucleotides, and that the presence of RNA in a cell increases retention of a corresponding antisense molecule.

HISM, SK-N-SH and NIH-3T3 cells were incubated for one hour or two hours with 5 μM of ¹²⁵1-c-myb phosphorotihioate sense or antisense oligonucleotides (S-ODNs) as described above. After incubation of the cells with the S-ODNs, radioactive medium was completely aspirated and the wells were gently washed with 1 ml of pre-warmed DMEM (37° C. /5% CO₂). The cells were then incubated with 1 ml of DMEM (37° C./5% CO₂) for 30 minutes (washout period) followed by 2 washes with cold PBS (washout kinetics).

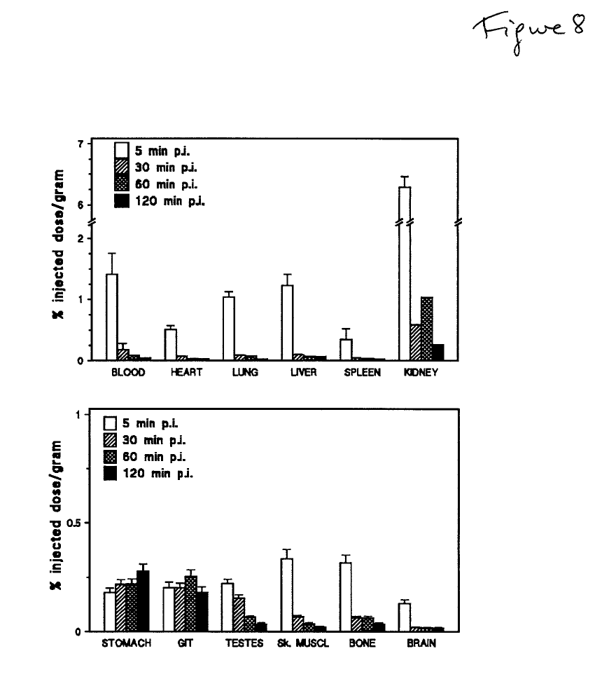

Absolute and relative retention of both c-myb sense and antisense sequences are presented in Table 1 and FIG. 7. For the HISM cell line, over 80% of the ¹²⁵I-c-myb phosphorothioate antisense radioactivity taken up following 1 and 2 hours of incubation was retained for 30 min. (data in Table 1 are presented as normalized cell bound radioactivity), whereas with the sense oligomer only 52% of the radioactivity was retained. These findings are consistent with results obtained with tritiated oligonucleotides. With the neuroblastoma cell line, ¹²⁵I-c-myb phosphorothioate antisense retentions were 66% and 82% for 60 and 120 min. incubations respectively. With the corresponding sense compound, retention was significantly lower; 38% and 44% for 60 and 120 min. incubations, respectively. Initial cell bound radioactivity was high in NIH-3T3 murine fibroblasts when compared with the human derived SK-N-SH and HISM cell lines. However, retention values (Table 1) were significantly higher in the human cell lines. Since HISM cells are known to express the c-myb mRNA sequence (Simons, M. et al., Nature, 1992, 359, 69-70; Simons, M. et al., Circ. Res., 1992, 70, 835-843), the higher intake and retention of the radio-labeled c-myb antisense, but not sense, by HISM cells relative to that in the other two cell lines, suggests that the presence of mRNA in a cell increases the intake and retention of constructs of the invention in such cells.

TABLE III
Percent retention of ¹²⁵I-c-myb sense and antisense oligonucleotides in
three cell lines following 1 or 2 hours of continuous incubation and a 30
min. washout period (see methods for details).

| | Percent retention of | | Percent retention of | |
|---|---|---|---|---|
| | c-myb antisense | | c-myb sense | |
| Cell Line | 60 min. | 120 min. | 60 min. | 120 min. |
| Human Intest. Smooth Muscle | 85.0 | 81.2 | 52.1 | 52.2 |
| (HISM) | | | | |
| Human Neuroblastoma | 66.2 | 81.0 | 37.8 | 44.2 |
| (SK-N-SH) | | | | |
| Murine Fibroblasts | 17.8 | 18.0 | 22.6 | 28.0 |
| (NIH-3T3) | | | | |

### Example 7

### Biodistribution of c-myb Antisense in Rats

CD Fisher rats, (175-225 g) were injected via the tail vein with 15-20 μCi radiolabeled c-myb antisense, (˜10 μg of c-myb antisense per rat). The rats were sacrificed by cervical dislocation at 5, 30 60 and 120 min after injection and samples of blood, heart, liver, kidney, muscle, stomach, gastrointestinal tract and brain were weighed, and radioactivity was measured with a well type gamma counter. To correct for radioactive decay and permit calculation of the concentration of radioactivity in each organ as a fraction of the administered dose, aliquots of the injected doses were counted simultaneously. The results were expressed as percent injected dose per gram (% I.D./g). Six rats were studied at each time point.

The results, which are presented in FIG. 8, indicate that the ¹²⁵I labeled c-myb octadecamer in rats exhibits rapid clearance from the circulation, which is primarily accounted for by renal clearance. Low levels of uptake were observed in quiescent tissues; bone, skeletal muscle and especially brain, in which the blood brain barrier further blocks accumulation. On the other hand, rapidly dividing tissue such as the gastric mucosa and gastrointestinal tract showed some accumulation of the radiolabel. These results are consistent with data published by Zamecnik et al (Proc. Natl. Acad. Sci. USA, 1994, 91, 3156-3160) indicating that cellular entry of S-OIDN's is related to cell cycling events. Free iodide is only a partial explanation for the accumulation of radioactivity in the stomach. Our results demonstrated that deiodination was only 2% of the activity in human serum after 4 hours incubation. The c-myb antisense oligonucleotide used in the present experiments contains a segment bearing four consecutive G residues, which in itself plays a role in bioretention within cells.

In conclusion we described: (i) a convenient and efficient method for derivatizing c-myb phosphorothioate for simple and rapid radioiodination. The radiolabeled product has excellent radiochemical stability in vitro and in vivo (ii) The ¹²⁵I-c-myb-phosphorothioates showed sequence specific uptake and retention in various cell lines. (iii) Human smooth muscle cells which are known to express complementary c-myb mRNA expression exhibited the highest level of uptake and retention, and validate the results of in vitro and in vivo studies with tritiated compounds and confirm the advantage of including an antisense molecule in a targeting complex (e.g., to increase uptake and/or retention in the cell). (iv) Biodistribution studies in rats parallel studies performed with tritiated oligonucleotides. The results show that the new radiolabeling procedures do not alter the biochemical properties of the antisense and thus may provide new tracers for diagnostic imaging and radiotherapy.

Equivalents

It will be apparent to those skilled in the art that the examples and embodiments described herein are by way of illustration and not of limitation, and that other examples may be used without departing from the spirit and scope of the present invention, as set forth in the claims.

1. A targeted oligonucleotide construct comprising:
a targeting moiety which localizes to a site in an organism;
an oligonucleotide complementary to a nucleic acid of interest; and
a detectable label.

2. A targeted oligonucleotide construct as in claim 1, wherein the targeting moiety is selected from a lipid, an antibody, a lectin, a ligand, a sugar, a steroid, a hormone, a nutrient, and a protein.

3. A targeted oligonucleotide construct as in claim 1, wherein the detectable label is selected from a chemiluminescent label, a radioisotope, a fluorescent label, a paramagnetic contrast agent, and a metal chelate.

4. A targeted oligonucleotide construct as in claim 1, wherein the oligonucleotide is selected from an antisense oligonucleotide and an antisense oligonucleotide analog.

5. A targeted oligonucleotide construct as in claim 1, wherein the detectable label and the targeting moiety are coupled to the oligonucleotide.

6. A targeted oligonucleotide construct as in claim 1, wherein the oligonucleotide and the detectable label are coupled to the targeting moiety.

7. A targeted oligonucleotide construct as in claim 1, wherein the targeting moiety and the oligonucleotide are coupled to the detectable label.

8. A targeted oligonucleotide conjugate comprising:
a targeting moiety which localizes to a site in an organism;
an oligonucleotide complementary to a nucleic acid of interest, and
a therapeutic agent.

9. A targeted oligonucleotide construct as in claim 8, wherein the targeting moiety is selected from a lipid, an antibody, a lectin, a ligand, a sugar, a steroid, a hormone, a nutrient, and a protein.

10. A targeted oligonucleotide construct as in claim 8, wherein the therapeutic agent is selected from an enzyme, an enzyme inhibitor, a receptor ligand, a radioisotope, an antibiotic, a steroid, a hormone, a polypeptide, a glycopeptide, a phospholipid, and a drug.

11. A targeted oligonucleotide construct as in claim 8, wherein the oligonucleotide is selected from an antisense oligonucleotide and an antisense oligonucleotide analog.

12. A targeted oligonucleotide construct as in claim 8, wherein the therapeutic agent and the targeting moiety are coupled to the oligonucleotide.

13. A targeted oligonucleotide construct as in claim 8, wherein the oligonucleotide and the therapeutic agent are coupled to the targeting moiety.

14. A targeted oligonucleotide construct as in claim 8, wherein the targeting moiety and the oligonucleotide are coupled to the therapeutic agent.

15. A method for preparing a targeted oligonucleotide construct, comprising:
forming a conjugate by connecting a targeting moiety which localizes to a site in an organism to an oligonucleotide complementary to a nucleic acid of interest; and
connecting a detectable label to the conjugate.

16. A method for preparing a targeted oligonucleotide construct, comprising:
forming a conjugate by connecting a targeting moiety which localizes to a site in an organism to a detectable label; and
connecting to the conjugate an oligonucleotide complementary to a nucleic acid of interest.

17. A method for preparing a targeted oligonucleotide construct, comprising:
forming a conjugate by connecting a detectable label to an oligonucleotide complementary to a nucleic acid of interest; and
connecting to the conjugate a targeting moiety which localizes to a site in an organism.

18. A method for preparing a targeted oligonucleotide construct, comprising:
forming a conjugate by connecting a targeting moiety which localizes to a site in an organism to an oligonucleotide complementary to a nucleic acid of interest; and
connecting a therapeutic agent to the conjugate.

19. A method for preparing a targeted oligonucleotide construct, comprising:
forming a conjugate by connecting a targeting moiety which localizes to a site in an organism to a therapeutic agent; and
connecting to the conjugate an oligonucleotide complementary to a nucleic acid of interest.

20. A method for preparing a targeted oligonucleotide construct, comprising:
forming a conjugate by connecting a therapeutic agent to an oligonucleotide complementary to a nucleic acid of interest; and
connecting to the conjugate a targeting moiety which localizes to a site in an organism.

21. A method for introducing a targeted oliognucleotide construct of claim 1 into a cell, comprising contacting a cell with a targeted oligonucleotide of claim 1, such that the targeted oligonucleotide is introduced into the cell.

22. The method of claim 21, wherein the cell is in vitro.

23. A method for treating a physiological condition in a patient, comprising administering an amount of a targeted construct of claim 8 sufficient to treat the physiological condition.

24. A method for imaging a physiological condition in a subject, comprising:
administering to the subject a targeted construct of claim 1; and
detecting the label in the patient.
